SEQUENCE LISTING (7 sequences)

SEQ ID NO: 1 - RNA, 1876 nt, Artificial Sequence
  Phlebovirus
acacaaagaggccgcuaauagaguacaggaagucaguaugacgucuuauagugagauccuugaggaguucggacugucugagauagacaacacucagcuagcccgaugggaggaacucuuugccuaugaggguuuugacccugucaaaaugcuggcaaagaugaagaagcuugccccugaugacuggagggaugauguaaaaaucaucauugucuuugccuugacaaggggcaacaaaaugagcaaggcugccaacaagaugagugauaagggcaagacugagcucaugcggcuuaaagccagguacaaucuaguggagaauccccagaguagggaugacaucacccccacuagaguugcaucugcucucccaaccuggacugucagagcggcagcagcccugaaggauucccucccuauggguccagcugccaucaaggcaaaugcagagaagaccaucccagcugagaugugcugugcugcauucgcuggcguaauccccacaagcgggauugccgaggaggauacugaggcuaucaccaaagccuucuuuguguggcaguacuacuucacacagcugaucaacccucagaugaggggaaagaagaaagaggugauuuucaguucuuuugaugcugccgugcgagcugccaugaacucugccagcuaccccaacaaaucccgcaucgacuuccuucagggucuugggaucuugaaccagaagaagaaagcgguggagucuguaagggcucuugcagagcucugggaugagauggaguaaaccuuucccacccaaauucaaucuggcuaaauuucaucagcuuuccaauuaggcuaaauuugccuuccucucccugccuuuaggcagggagaggggggcuuagaggggaaucaaaagucauagggaucagguggcggcagguagagcuggccaguccccaccgaaggaggugcugaaguaauucaaccugaaugaguccucagucuccaagaagaauaguaggcucuccguugaggauaggguuucaagaaauuccuucugaucuccaggaggccuacuaacucuccgacgaucaccagaaugauaagucaucaagagggaagaauaagggucaaaagggucaucuuccuuaacccucagauauucaucaacaucucgucucauucgcuuggggagcuuccuaggauagaaccagcauaagguuugcaguaucucauaaacugaagggccgacagagcaguaagggaccauaaagaggucccuuguggcugcccuugcaaaggacagauauugcagagcugccacguaauagaugauugaaucaguugguauugauguugggcaaauaccaagcugcuuagcucuccuaaaacacuucucauaaagaaugaugauucugucagcgguggugcagccaggaggagcaucccauguggaccucuucaggagcuuaaaaaaugccuuccaagacaaacacuguucaacugggguuucaaaguugaagauccgcacugcuagagacauccagcuugucguggccuuuccaguaggccaccucagugaagaccuuugcuccugaguccaucccaacccaugugguaugcaugacauaaucgccgagaucagaagguacggcuuaggcaucugccugaagaugcuagauccauccacaaaguagcaguggcugaagaaugugccacucaggcuaggguaagcaacccccauggaucggaauaauguuuucagaaucccuucccuggcacgagccuuacuagcccuccaguaaccuccaaagaucaguagauugucauccacugguucaaacaaaguagguaguuuugguacugagucauccauggugcggccucucugugu

SEQ ID NO: 2 - RNA, 3313 nt, Artificial Sequence
  Phlebovirus M segment
acacaaagacgccagcuacuagcagagauguucccaacuuugagacucuuggcacuugcccugauaguugcggcccaugggggagaccuacccugcgacagagacuggauaaaagagggaaaaaaugaagaguacaucaagaacucugugaacuugucaagcacagagggguucagcuacucagucaagcuuugcuugaugagccuuggccaucacuggcgacuuaucauagcucaagaucacaguguugaagaaaaggcuguagauuuccuauuaccuucuagccagggauggagagggaaugagcacacaaggggagucacccuguuucacccggacaucauagauggauucuucuuagguugguuugacacuaaauuuuaucucuacagugagguugcagaaaggcuaccaguuccuccuggcugccauaaagcuaagcuuugcuauuucaugacuauagaagggcccaugaggauagacugugccgcuaagcagaagggagugggaaaaacuggacacugcaaugcaaaggcuuucucauuagacccucacaaugugagagcccucggaguacagaggucucagccaggcacgguccucucagagacccagaaagacuaugucauaagugagagaucagcugccaacccugguguuugcacuauugauggaucuguaaucaaucacugugagagaaaauacccgggcuccuggaaacagaugcauuacgcuuucuuccaaggcccagaggguggacugacauuugucgcccccacucucaauguaguguggaugcaagauccaaggcugagcugggcauauuauugcaaaaacauuucccuacaccaccuggaucauauaaacccuugcaggcagaauugcacgaaggagacuugccaaggagacgaccucuuuugucaugagcaaggauguacacacgacaaggcaugucauugccagcaccuucccacuuucggcauggccaagguccuuguaggugaaucauggcugaucccauuugugguggggguucaagaauuuuuugugguagaagaacuacuaggaaauccucucuugccuagaaaguguucuacuuguucucucacauguacaagugagggcauaucguuccacucaauaaaagaugagcucaaagaaaucacggucuguauagaaggauccugcuccucccggcaaggaguuggcggcaagguauggagcaucccauuuggaaaccaaaugccuagggguggagcacaagcaguggcccaagggcuaaccauggcuggagaggcauucagccuaucugcagacugugggguuagaucaggaugugaccaaauaaauugcaucuucugcacggacaugcugagcaacccacaauguuauccguauggaaaaugguuguuauugugcauuauggcaggggccuuguauuuuguccugucucuaagcaaguuugcacuccagaugcuacuaaugcuagcucaguuacugaccucuccccugaugcuaguauggaaaguuucaaaaugucuaggacgcuuaggcagaaggaggacaaggaggacauacaccagacugcugcaggcuauagaagagggagaugcagaaccggacaucacugcugacgucaagagaguugacaaaccaaagaaaaugccuagaauuguuguuuucgcauugaugcuucucacugugguaggugcaaccuuggccugugaugaaacccaccuaauuucagagacaucuguggugugugaugccacaagagcaucccacuacaugugcaccacaaaagagauugugguucucaaaaaucucagggcgggaaaaaccaucugcauuagcaucaaggggccagguggggugaucucaaaacccuuaaaaauuguuauggaggacauuguggggagggccgaccugcuagaugcuuauuacacauucacugggcaggccaacugcaagucugucaggaggugcaggugggcaggcacaugcagcucugguugcuccggaguugauaagagcucauuugugcaggagcuaggggcuaguggguugcauucucaccccaacuggcuggacuguuaugauggcuguggaggagcagcuuguggguguuucaacgcagcaccaagcuguauuuucuugcagagauauguaaccaacaaggaugccagaguuuacaagguguuuaaaacgucugccugguuuuuaaauacccgugcaagagucucuguagaugaucgaacaucuggggucacucuuaaagauggagaaacaaagacaauaggaaaagucaguuuuagcuauaggacugacagaaaccuauuugcaggucucucgcucccuccaauugugucagagauaaaggccucugguaagcccaaugauuuccucuuugaaaaccaagguagccacccuaaaugcaaagaccagaauucagccauacugagcagcucuucaggguguauaguggaccaaaauagcaucacagugaaucccagaguugaugauguguccugccgcuccaaucuugucucauuuaauggaauggaaacccuaaaaccacuaccccaaagagugggagauuucuuggucagcuucaaaggcucagaaccuguccuucuagcaguuggcagcacuaauguuguggagggggaacuaagaguugacaugucacucaaaaacuucaagauacauguggauggaaacauaugcaggggcacauuagugucacucaagggcuguguaggcuguucacauggggcuacugccaaccugaaaauccacgcugauaaagcuggagaguccucccuagaaugcccaagcagcagcuguagauuaaagcuagagaagggggugaaugacuuggagugcucccuaagguuuagcacuccaaacguggaugaaacuuguguuuuggucugcucagguuccaaggagcacuugcaggucacuggugcucugcuaaucgggggggacuucaagaaauugacagaugacucugcaucuaacuucgaacauucagauucaaaaacagcagggauccauauagggacuggcauuggaaacuggcuguccucacucuuuggauuuagugucaccagauggaucauuggaauagucgguuucauccuuacuuuuguuguucuggugguuugccugaaauggcuuuuguucaggguaugucggcgcucccagccagaaagugacaaggauaagaucccuuaacacuauucugguucuugcacuagucugugcccucacuuagguggggguucaggaaugcuggcggucuuugugu

SEQ ID NO: 3 - RNA, 6341 nt, Artificial Sequence
  Phlebovirus L segment
acacaaagacgccaacauggaaacuaggauacggaaugaggugaucccguuccccaggaggaacaacaucacugcugagggcuugguguaucuugcccaccuugacgacucuagacucccagaauucagcaucacaccccaggaggacucucuucuucucgagauagaugagaucccagaaaguaucagucaagugggcagcagcauaauuguggggcaccaacgguuugaggucggaaagguuagcacgcuaauucaugauuuuacuuuugcagccauugcugaagaaacugauaagcccuuaucaagacauuuuccaaucauaaaugauggugaggaccaucuaacaccagaucucaucauugaugcuuccgccacggagaagagggugauagaguuugcaacucacagaggcccaggggugagggguguguuaaacuauuacaaucaaaaaauugaaaaguacacaagaccacuggcagcaaggcaggaaagaacacaaugggacauaucuuugucuuguauugcaguaggguacgaccauguggugucagaucucacacuaucacaagccacuguugaugaacuaguguacagauucagauuugcuucugcuguuuucaaucagauccuuguagcuuucccagaacuagcuccaaaagucucugaggaaauaucuucggcugagagagcuggaagagaaugccugaacuucuugacaagaaccuucauagaggaaccugaaggcgagagccuccuagagaacuuuccucuaggguccaaaagauauuuggaugcuuuccuugagaauuccgaagaugagcaguaucucaagaggguuuucgcagggacuuuccaagaaacccuagacgagauaaggggagaacacauucuagaaggacacaggaggggcccagccuuggagagaaauggugaggaagcaucaucacaaauggcacaacuaguuaaggaucaacuugaugcuuaccuaaaacagaaagugaagcaggaugagaguaaauucaaaucaacuaucaagauuccuggcuggcugauuccagacacaccgagaaccauugauauaggcauaaaccacaaggaucuagaaggcaugucugaugaaacugucacagcuagaguuuggaaggcagcucuaacugaggucaucuucacuccucagaacagcuggcaagaggaccuugagagagaauaugcaauagcuaccaguaggcugcagguugacccagagggggaaaacaccaaacaagacuaccauagagugaaaauaaaucucccugcucaagacugggugucucuugcugccagaggcuuagaagggaaguccaugagacaagaagagcucgucagaaaaacugaugaggcaucccauacuguccugagcccacaccaugacaucucuggcauuaguuccugcauugagucaaacaacugguuccaccacuucaaguuaggacaggaagaaaaccucuacgggguucuggauguccugauaucaucagcaauggaaguagccacugauggucaacaagugggugaaaagauucugaaggcuuucaaggauuacagaaaccucaacuguguucauugggcaagcaugguuacccagaucgcaacagagcuagccauugcugcaaaacaaagcugcaaaagacaucacuucaucauaaaaaaaguccccaacuuccccauauauauguugaucaaaaccagcaggucaggaucucauguauucuacagcuuagcugccaggaccaacucugucaagcacgagcucucaggcagagucuucaaagaguacgagcguucagggaacugguuuauaacugaguucaauucuguuaaguugugcaagguggagaacaugcucaaguugccuauguugauuguugcccugauuucauacucacaagagucagacgaaaaaguggucaccaaacugucuagaccaaccacauucacaagaaagcuagugaccacagccauuaugguguguauggaggauaaggccuuaacugaggaauugauuaccauacagagauacauccagauggagggguuugugacagaaccucuacugcccaagccccacaagauguuggagaagcucucuguaccacuccgaacuacacugcaaguuuuccucuuucagcagugucucaaggcgaucagauccauuucaagaaccccuuauaggaggaugugcaaggcaggcgagguguccuggagagguugcuucucuggugucaucgggauguccugcuccccugagcagauggugaacucaugguacuugggguacuuaaagaauaaagacgaggauacagaaguaaacaugcugggugcaauguaugagaagauugugaaaguugaagaaaaaaggccccuggacuccucuaaccugcuagguggggacccauuaaaacccaacaugcaugaauucucaggcuccuucaucaaauuuauggcgcgugcucuaaaugaugagauucgaggucguaaagggcacaacuggcaggaagagauuacaacccaauucuaucgaucuauugguucuguaacccuugaccaauuagcaacuuugaaagccucaagcucuuuuggcccagaaugggaaaacuuugaugauguuaggcuaaaggaauaccacagggucaaggugcuagagagaaucucugaacugguccaggaaggccauaccuucuacacagacacaauaggccaauguuuuagaguaguucuugaagaugggugcaugagaauuugcuuguucaaaaaagcacaacaugguggacugagggagauauacgucuugagacuggaagagagacucauccaauuugggauugaguuacuagcucgcaaaauaaaugaggcuguuggucaugagaccaucagugugccaaguagaaaagaagaaauucuggacacucaucaagccagagcugcaaaagcauguggugaaggcacacucaucacugucuguaguucuaaugaugccagaacuuggaaucaagggcacuacaccacaaaauuugccuuucucaugugugagcucaugccucgagaacugcacggcuucaucuggaugucuugugcuaucuucagaaagaagaagaugaugcuaaaccuggacuaucuaaauagcaugugcagaaagcggccugaggccaguggguucaaacaaagacuuuacgaugguuucucugggaaugaggaaguucccuggauacagaaagggaaaaccuucauccaaacagagacagggaugaugcaaggaauuuugcauuuaaccucuagcuuguuucaugcagccuaucaagcggcuaucaggaaaauuuuaaaggcaaaacugaaaacaacaaucaacagcagagucuugauagaugugauugaaggcucugaugauagugcaauugugauaucugcagcugucaagagugaugacgaagaaagaaaauuuaggcugauggcuucaguuuugcugcacuggguuaaaaggcuuggccuuuaugccggaauuuacaugucccccaaaagcacaauugguugcauugaugugugcgaguacaacucugaguuuagauuuuccagacucauguguaggcccacccucaagugggucacagcaugucuaaacauuccagaaguugagaaaauagcagacaggcaagagucauucucuaauuuguugaccucuguucuagaaggagguggaacaucaaguuuauguucaguucuacaacuaacacaggcauggugucacuauguccuucugggccuauggucauccuguguguuuaagcugauagcgccuuacauggaagagucaaagaauccugacauaggcuuuuucauucuggauugcccagucucagcagggauuuuagguuuuagacacaaccucuggagguauguuagaaaaaccaaguuaucagcaaucuuugccaaaauacuagacaauaagggucaugaaggcuucacucucaccucaggagggucucucuccaaaugccauuugaucagauggggagacagaaagaaacuagacgguauuaagaaccgaacagguaugaacuuagacuggcggauauuaacagaugaggaccccucucuucuguacaaaccaccucagaacaaucaugaggugaagguggccaucagucuuaaacuagauaguccaggaguugcagagaguuugucuaaaggcaauguucuuggcagaguuauagcaucaucaguuuacauuuuacagagacguugcaucacaguaaggagccagaggaggaaguacacccuugcagaacugaugcuagagacagaagauguugggaggacuuuaacucuagcugaagagaaugccuuguuuggugauauuaucgcauuggagaagaaugaggcccugugucaauccuauggagaggcaauugggaacuuuauaggcaaguccagagagaugaggagggccaagguugaaguccuauccgcugacgaagcauuuagagcaccuccuguaaagaucaugggggaugucuucuuuggaacaacuaaaagccacauggggcaaaacauguuggccagggaacuugguuaucuuaaagaaagcuucccauggauaucugcagacccucaugagugcuuaaguaacucuccauucaccugucaggccgaguugaaaacuuucuuugagaaacuagaacagaagacuaggaagguuaggaugauaggugcugcuguuuucacaaggaugggucaaaccagcuuggauaaccuauugagauucaacuuccaaaaaaacuuugaacuaacucgacaggggaauggcgaguacgaagugguaaacgaagacgaaaaguuuugcaaacaugucauaacuaugauccuaucaggccccuucucaaacgagagaaagucacagaugauuguggauuuccucaagauaagugaccucuccccucccaagacccuucuuaaaaaaagcagaaccaaugucuugagggucaucagagcuuggcuagaaggccauuccagaauugaagagcucguugagagaagcauggauggcauauguggagccuuuacugucagacagaaaaucagcagggauagcaguggucaacuccaguauaggggacugggggucuggacuggaagaauagaggguacagacuguagacuacaaauagagaacuccgguccaggggaacaaguuuuaaagcaaguuaccaucuccucagacaaaaauuuaucugauuuccugucuggucuggaacgccucugcaaagaauuaagaguuaccaacccaaggaaauccugggcaaagcagaagaagcaggauuugagccugauaggagcucuuguugacuuuaaacuugaaucacgacucucuugugcgggcuguccucugaggcuuguaccugcaucugaggguuuucugaaagagaugauggauagguccaacauuucucuaauuguaagaggugacauucucaacaucaaagcagacuuaggagauggcagacuuaucaccaugauaaguuacaaggcaucuggucaggaugcagaccaagagcaaggagcuagauuuuucucccgaagagaagugcuuaagacacaccuaucauggucaguuagggagccauaccuuagcuggaucaguuugcggccagucccggaugccuuaguucccagauuagaagaggagaucaugggaaagagaagaacugaaggcauugaugcaaaacaacucaaggacgucuaccaaaggugcugcaaaaguuccuuaagacgaaagggccucacuguuggucaguucucaaccgugaaacucaggauugaagacacuagugaugauguggagaucaaucuucuggagcuauuagaagaagaaguugauuuuacauccaccuuagagaggacuucucuugauaugcuagaggacucaaucgaggauauggagguauucuucggugaggaagacuuagaaauuuuugauuucacagagcucauaucucgugcagauccacuagccuaccauggcuuuugugaccgcauuauagaagacuucgugaagaaauuaggacaugaugucauacggagagccauccaaaagagagagaugcuucagacagaugccgagcuugugaagaagcuuuuugauugcauaggcgagaacccugaagaacuagagauuguccacgaugagcuggaugaguaugcauccucuggccaaauaacagaggaggucugggguuagugaucacucuuaccgugucucuccugggaauucaggggagguuaucugguguuggcggucuuugugu

SEQ ID NO: 4 - protein, 247 aa, Artificial Sequence
  Phlebovirus nucleocapsid
MTSYSEILEEFGLSEIDNTQLARWEELFAYEGFDPVKMLAKMKKLAPDDWRDDVKIIIVFALTRGNKMSKAANKMSDKGKTELMRLKARYNLVENPQSRDDITPTRVASALPTWTVRAAAALKDSLPMGPAAIKANAEKTIPAEMCCAAFAGVIPTSGIAEEDTEAITKAFFVWQYYFTQLINPQMRGKKKEVIFSSFDAAVRAAMNSASYPNKSRIDFLQGLGILNQKKKAVESVRALAELWDEME

SEQ ID NO: 5 - protein, 316 aa, Artificial Sequence
  Phlebovirus nonstructural protein
MDDSVPKLPTLFEPVDDNLLIFGGYWRASKARAREGILKTLFRSMGVAYPSLSGTFFSHCYFVDGSSIFRQMPKPYLLISAIMSCIPHGLGWTQEQRSSLRWPTGKATTSWMSLAVRIFNFETPVEQCLSWKAFFKLLKRSTWDAPPGCTTADRIIILYEKCFRRAKQLGICPTSIPTDSIIYYVAALQYLSFARAATRDLFMVPYCSVGPSVYEILQTLCWFYPRKLPKRMRRDVDEYLRVKEDDPFDPYSSLLMTYHSGDRRRVSRPPGDQKEFLETLSSTESLLFFLETEDSFRLNYFSTSFGGDWPALPAAT

SEQ ID NO: 6 - protein, 1070 aa, Artificial Sequence
  Phlebovirus glycoprotein precursor
MFPTLRLLALALIVAAHGGDLPCDRDWIKEGKNEEYIKNSVNLSSTEGFSYSVKLCLMSLGHHWRLIIAQDHSVEEKAVDFLLPSSQGWRGNEHTRGVTLFHPDIIDGFFLGWFDTKFYLYSEVAERLPVPPGCHKAKLCYFMTIEGPMRIDCAAKQKGVGKTGHCNAKAFSLDPHNVRALGVQRSQPGTVLSETQKDYVISERSAANPGVCTIDGSVINHCERKYPGSWKQMHYAFFQGPEGGLTFVAPTLNVVWMQDPRLSWAYYCKNISLHHLDHINPCRQNCTKETCQGDDLFCHEQGCTHDKACHCQHLPTFGMAKVLVGESWLIPFVVGVQEFFVVEELLGNPLLPRKCSTCSLTCTSEGISFHSIKDELKEITVCIEGSCSSRQGVGGKVWSIPFGNQMPRGGAQAVAQGLTMAGEAFSLSADCGVRSGCDQINCIFCTDMLSNPQCYPYGKWLLLCIMAGALYFVLSLSKFALQMLLMLAQLLTSPLMLVWKVSKCLGRLGRRRTRRTYTRLLQAIEEGDAEPDITADVKRVDKPKKMPRIVVFALMLLTVVGATLACDETHLISETSVVCDATRASHYMCTTKEIVVLKNLRAGKTICISIKGPGGVISKPLKIVMEDIVGRADLLDAYYTFTGQANCKSVRRCRWAGTCSSGCSGVDKSSFVQELGASGLHSHPNWLDCYDGCGGAACGCFNAAPSCIFLQRYVTNKDARVYKVFKTSAWFLNTRARVSVDDRTSGVTLKDGETKTIGKVSFSYRTDRNLFAGLSLPPIVSEIKASGKPNDFLFENQGSHPKCKDQNSAILSSSSGCIVDQNSITVNPRVDDVSCRSNLVSFNGMETLKPLPQRVGDFLVSFKGSEPVLLAVGSTNVVEGELRVDMSLKNFKIHVDGNICRGTLVSLKGCVGCSHGATANLKIHADKAGESSLECPSSSCRLKLEKGVNDLECSLRFSTPNVDETCVLVCSGSKEHLQVTGALLIGGDFKKLTDDSASNFEHSDSKTAGIHIGTGIGNWLSSLFGFSVTRWIIGIVGFILTFVVLVVCLKWLLFRVCRRSQPESDKDKIP

SEQ ID NO: 7 - protein, 2085 aa, Artificial Sequence
  Phlebovirus RdRP
METRIRNEVIPFPRRNNITAEGLVYLAHLDDSRLPEFSITPQEDSLLLEIDEIPESISQVGSSIIVGHQRFEVGKVSTLIHDFTFAAIAEETDKPLSRHFPIINDGEDHLTPDLIIDASATEKRVIEFATHRGPGVRGVLNYYNQKIEKYTRPLAARQERTQWDISLSCIAVGYDHVVSDLTLSQATVDELVYRFRFASAVFNQILVAFPELAPKVSEEISSAERAGRECLNFLTRTFIEEPEGESLLENFPLGSKRYLDAFLENSEDEQYLKRVFAGTFQETLDEIRGEHILEGHRRGPALERNGEEASSQMAQLVKDQLDAYLKQKVKQDESKFKSTIKIPGWLIPDTPRTIDIGINHKDLEGMSDETVTARVWKAALTEVIFTPQNSWQEDLEREYAIATSRLQVDPEGENTKQDYHRVKINLPAQDWVSLAARGLEGKSMRQEELVRKTDEASHTVLSPHHDISGISSCIESNNWFHHFKLGQEENLYGVLDVLISSAMEVATDGQQVGEKILKAFKDYRNLNCVHWASMVTQIATELAIAAKQSCKRHHFIIKKVPNFPIYMLIKTSRSGSHVFYSLAARTNSVKHELSGRVFKEYERSGNWFITEFNSVKLCKVENMLKLPMLIVALISYSQESDEKVVTKLSRPTTFTRKLVTTAIMVCMEDKALTEELITIQRYIQMEGFVTEPLLPKPHKMLEKLSVPLRTTLQVFLFQQCLKAIRSISRTPYRRMCKAGEVSWRGCFSGVIGMSCSPEQMVNSWYLGYLKNKDEDTEVNMLGAMYEKIVKVEEKRPLDSSNLLGGDPLKPNMHEFSGSFIKFMARALNDEIRGRKGHNWQEEITTQFYRSIGSVTLDQLATLKASSSFGPEWENFDDVRLKEYHRVKVLERISELVQEGHTFYTDTIGQCFRVVLEDGCMRICLFKKAQHGGLREIYVLRLEERLIQFGIELLARKINEAVGHETISVPSRKEEILDTHQARAAKACGEGTLITVCSSNDARTWNQGHYTTKFAFLMCELMPRELHGFIWMSCAIFRKKKMMLNLDYLNSMCRKRPEASGFKQRLYDGFSGNEEVPWIQKGKTFIQTETGMMQGILHLTSSLFHAAYQAAIRKILKAKLKTTINSRVLIDVIEGSDDSAIVISAAVKSDDEERKFRLMASVLLHWVKRLGLYAGIYMSPKSTIGCIDVCEYNSEFRFSRLMCRPTLKWVTACLNIPEVEKIADRQESFSNLLTSVLEGGGTSSLCSVLQLTQAWCHYVLLGLWSSCVFKLIAPYMEESKNPDIGFFILDCPVSAGILGFRHNLWRYVRKTKLSAIFAKILDNKGHEGFTLTSGGSLSKCHLIRWGDRKKLDGIKNRTGMNLDWRILTDEDPSLLYKPPQNNHEVKVAISLKLDSPGVAESLSKGNVLGRVIASSVYILQRRCITVRSQRRKYTLAELMLETEDVGRTLTLAEENALFGDIIALEKNEALCQSYGEAIGNFIGKSREMRRAKVEVLSADEAFRAPPVKIMGDVFFGTTKSHMGQNMLARELGYLKESFPWISADPHECLSNSPFTCQAELKTFFEKLEQKTRKVRMIGAAVFTRMGQTSLDNLLRFNFQKNFELTRQGNGEYEVVNEDEKFCKHVITMILSGPFSNERKSQMIVDFLKISDLSPPKTLLKKSRTNVLRVIRAWLEGHSRIEELVERSMDGICGAFTVRQKISRDSSGQLQYRGLGVWTGRIEGTDCRLQIENSGPGEQVLKQVTISSDKNLSDFLSGLERLCKELRVTNPRKSWAKQKKQDLSLIGALVDFKLESRLSCAGCPLRLVPASEGFLKEMMDRSNISLIVRGDILNIKADLGDGRLITMISYKASGQDADQEQGARFFSRREVLKTHLSWSVREPYLSWISLRPVPDALVPRLEEEIMGKRRTEGIDAKQLKDVYQRCCKSSLRRKGLTVGQFSTVKLRIEDTSDDVEINLLELLEEEVDFTSTLERTSLDMLEDSIEDMEVFFGEEDLEIFDFTELISRADPLAYHGFCDRIIEDFVKKLGHDVIRRAIQKREMLQTDAELVKKLFDCIGENPEELEIVHDELDEYASSGQITEEVWG

(19) United States
(12) Patent Application Publication
(10) Pub. No.: US 2016/0083698 A1
(43) Pub. Date: Mar. 24, 2016

(54) LONE STAR VIRUS

(71) Applicants: THE REGENT OF THE UNIVERSITY OF CALIFORNIA, Oakland, CA (US); THE UNITED STATES OF AMERICA, AS REPRESENTED BY THE SECRETARY, DEPARTMENT OF HEALTH AND HUMAN . . ., Bethesda, MD (US)

(72) Inventors: Charles Chiu, San Francisco, CA (US); Andrea Swei, San Francisco, CA (US); Barbara J.B. Johnson, Fort Collins, CO (US)

(73) Assignees: THE REGENTS OF THE UNIVERSITY OF CALIFORNIA, Oakland, CA (US); THE UNITED STATES OF AMERICA, AS REPRESENTED BY THE SECRETARY, DEPARTMENT OF HEALTH AND HUMAN . . ., Bethesda, MD (US)

(21) Appl. No.: 14/785,268

(22) PCT Filed: Apr. 18, 2014
(86) PCT No.: PCT/US2014/034684

§ 371 Date: Oct. 16, 2015

Related U.S. Application Data
(60) Provisional application No. 61/816,634, filed on Apr. 26, 2013.
(60) Provisional application No. 61/814,181, filed on Apr. 19, 2013.

(51) Int. Cl. C12N 7/00; A61K 39/12; C07K 14/005; C07K 16/10; G01N 33/569; C12Q 1/70

(52) U.S. Cl. CPC C12N 7/00; G01N 33/56983; C12Q 1/701; C07K 14/005; C07K 16/10; A61K 39/12; C12N 2760/12221; C12N 2760/12234; G01N 2333/175; G01N 2469/10; C07K 2317/24; A61K 2039/5254

(57) ABSTRACT

This disclosure concerns the isolation, identification and sequencing of a unique Phlebovirus, the Lone Star Virus. Lone Star Virus nucleic acid molecules, proteins and antibodies are disclosed. Methods are also disclosed for the detection, diagnostic and treatment of the Lone Star Virus.

### CROSS REFERENCE TO RELATED APPLICATIONS

This claims the benefit of U.S. Application No. 61/816,634, filed Apr. 26, 2013, and U.S. Application No. 61/814,181, filed Apr. 19, 2013, which are both incorporated herein by reference in their entirety.

### ACKNOWLEDGMENT OF GOVERNMENT SUPPORT

This invention was made with government support under grants no. R56-AI089532 and R01-HL105704 awarded by the National Institutes of Health (NIH), a research grant from the National Research Fund for Tick-borne Diseases, and funding from the Center for Disease Control. The government has certain rights in the invention.

### FIELD

This disclosure relates to the isolation, identification and sequencing of a unique Phlebovirus, the Lone Star Virus, and the use of the Lone Star Virus (LSV) nucleic acid molecules, proteins and antibodies in detection and therapy.

### BACKGROUND

Bunyaviridae is the largest family of viruses, with over 350 species that infect a broad range of hosts including plants, arthropods, and vertebrate animals (Walter and Barr (2011) J Gen Virol 92: 2467-2484). Viruses in the family Bunyaviridae infect a wide range of plant, insect, and animal hosts. The Bunyaviridae family is comprised of five genera: Nairovirus, Bunyavirus, Hantavirus, Phlebovirus, and Tospovirus (Guu et al. (2012) Adv Exp Med Biol 726: 245-266). Their genomes consist of three single-stranded negative-sense RNA segments: large (L), encoding the L protein, an RNA-dependent RNA polymerase (RdRp); medium (M), encoding glycoproteins Gn and Gc; and small (S), encoding the nucleocapsid protein (N) as well as an ambisense nonstructural protein (NSs) in a subset of viruses.

Recently, emerging bunyavriuses, putatively tick-borne, have been discovered in association with human acute febrile diseases, including Severe Fever with Thrombocytopenia Syndrome virus (SFTSV) in China and Heartland virus in the United States. Bunyaviruses pathogenic to humans are also associated with respiratory, and hemorrhagic diseases. These bunyaviruses include Crimean-Congo Hemorrhagic Fever (CCHF) virus (Ergonul, (2012) Curr Opin Virol 2: 215-220, a tick-borne acute hemorrhagic disease in Asia, Europe, and Africa with a case fatality rate of up to 30%, and hantaviruses (Jonsson et al. (2010) Clin Microbiol Rev 23: 412-441), a suite of rodent-borne diseases worldwide that are associated with pneumonia or hemorrhagic fever with renal syndrome.

In 2011, a new bunyavirus in the Phlebovirus genus, named Severe Fever with Thrombocytopenia Syndrome Virus (SFTSV), was reported as the cause of an outbreak of severe febrile illness in China (Xu et al. (2011), PLoS Pathog 7: e1002369; Yu et al. (2011) N Engl J Med 364: 1523-1532; Zhang et al. (2012) Clin Infect Dis 54: 527-533). Between 2008 and 2010, approximately 500 patients from eastern China, predominantly farmers in rural hilly areas of Hubei and Henan provinces, were diagnosed with SFTSV infection. The disease caused by SFTSV was characterized by fever, anorexia, fatigue, and depressed platelet and white cell counts (Zhang et al., 2012, supra). Because of the similarity of the clinical symptoms of SFTS disease to those seen in human granulocytic anaplasmosis, the etiological agent was originally believed to be Anaplasma phagocytophilum. A novel Phlebovirus was identified as the cause of SFTS (Xu et al., 2011, supra; Yu et al., 2011, supra) and additional findings implicated the hard tick, Haemaphysalis longicornis, as the vector of SFTSV (Zhang et al., 2012, supra). Heartland virus (HRTV) is a tick-borne phlebovirus distinct from LSV and associated with two human cases of critical febrile illness from Missouri, was also reported (McMullan et al. (2012) N Engl J Med 367: 834-841). Strains of Bhanja (BHAV) and Palma (PALV) virus have also been fully sequenced and found to constitute a novel clade of tick-borne phleboviruses (Dilcher et al. (2012) Virus Genes 45: 311-315; Matsuno et al. (2013), J Virol.). Although the pathogenic spectrum of the BHAV group viruses has not yet been fully defined, Bhanja virus has been associated with febrile illness with central nervous system involvement in both laboratory and naturally infected cases (see, for example, Calisher and Goodpasture (1975) Am J Trop Med Hyg 24: 1040-1042; Punda et al. (1980) Zentralblatt fur Bakteriologie: 297-301; Vesenjak-Hirjan et al. (1980) First natural clinical human Bhanja virus infection. Zentralblatt fur Bakteriologie: 297-301.

A need remains for agents that can be used for the detection and treatment of Phlebovirus infections.

### SUMMARY

Disclosed herein is the isolation and identification of a previously unidentified Phlebovirus. In particular, disclosed are Phlebovirus isolates from the lone star tick, Amblyomma americanum. The sequences of the genome for this Lone Star Virus (LSV), as well as the amino acid sequences of the proteins encoded by the virus, are further disclosed.

In some embodiments, an isolated Phlebovirus is disclosed that includes an S segment, an M segment and an L segment. wherein: (a) the nucleotide sequence of the S segment is at least 80% identical to SEQ ID NO: 1; (b) the nucleotide sequence of the M segment is at least 80% identical to SEQ ID NO: 2; (c) the nucleotide sequence of the L segment is at least 80% identical to SEQ ID NO: 3; or (d) all (a), (b) and (c). The Phlebovirus can be attenuated.

In some embodiments, an isolated nucleic acid molecule is disclosed that includes a nucleic acid sequence at least 90% identical to an open reading frame of SEQ ID NO: 1, the complement thereof, SEQ ID NO: 2 or SEQ ID NO: 3. The isolated nucleic acid molecule can include an open reading frame of the nucleic acid sequence set forth as SEQ ID NO: 1, the complement thereof, SEQ ID NO: 2 or SEQ ID NO: 3. The isolated nucleic acid molecule can be a cDNA. Oligonucleotides, such as primers and probes, are disclosed that are specific for these nucleic acid molecules. Polypeptides are also disclosed that are encoded by these nucleic acid molecules. These polypeptides include, but are not limited to, polypeptides comprising the amino acid sequences set forth as SEQ ID NOs: 4-7.

In additional embodiments, isolated antibodies, or antigen-binding fragments thereof, are disclosed that specifically bind the LSV disclosed herein, or a polypeptide encoded by the LSV.

Methods are also disclosed for detecting a LSV, LSV polypeptide, LSV nucleic acid molecule or an LSV-specific antibody in a biological sample. The present disclosure further provides a method for identifying a subject infected with LSV. Thus, the use of antibodies, oligonucleotides and polypeptides to detect an LSV infection is provided by the present disclosure.

Immunogenic compositions are disclosed that include a recombinant LSV, a LSV nucleic acid, and a LSV polypeptide. The use of these immunogenic compositions to elicit an immune response against LSV is provided by the present disclosure. Thus, methods are provided from the production of antibodies. The compositions disclosed herein can be used to induce an immune response to LSV in a subject, such as a healthy subject or a subject infected with the LSV.

The foregoing and other objects, features, and advantages of the invention will become more apparent from the following detailed description, which proceeds with reference to the accompanying figures.

### BRIEF DESCRIPTION OF THE DRAWINGS

FIG. 1. Time course of the development of cytopathic effects by Lone Star virus in human (HeLa) and monkey (Vero) cell cultures. CPE is shown at 24, 48, 72, 96, and 120 hours post-inoculation (hpi). Uninfected controls at 120 hpi are also shown.

FIGS. 2A-2D. Identification and assembly of the LSV genome by unbiased deep sequencing. (A) Using a rapid computational pipeline, reads identified as bunyaviruses by SNAP nucleotide alignment (light grey) or RAPSearch amino acid alignment (dark grey) were mapped to the assembled LSV genome. The coverage (y-axis) achieved at each position along the genome (x-axis) is plotted on a logarithmic scale. (B) De novo assembly of the LSV genome using the PRICE assembler (3 rounds of 15 cycles each) and LSV seed sequences (“S”) identified from (A). (C) The genome structure of LSV. Boxes represent open reading frames (ORFs) corresponding to the RdRp, G, N, and NSs proteins, flanked by noncoding regions, which are indicated by lines. Coding directions are indicated by arrows. (D) Mapping of the actual deep sequencing reads derived from LSV to the final assembled genome. The coverage (y-axis) achieved at each position along the genome (x-axis) is plotted on a logarithmic scale. GENBANK® accession numbers are reported in the text. Abbreviations: kb, kilobases; bp, base pairs.

FIGS. 3A-3B. Amino acid phylogenetic analysis of the four LSV protein sequences relative to those from representative phleboviruses and Gouleako virus. For the RdRp, glycoprotein, and N protein, Gouleako virus is included as an outgroup to the phleboviruses (tan). Gouleako virus, the closest known bunyavirus relative to phleboviruses, is a member of a proposed new genus in the family Bunyaviridae (Marklewitz et al. (2011) J Virol 85: 9227-9234). Also shown color-coded are the Uukuniemi, Bhanja, and SFTS clades of known tick-borne phleboviruses. GENBANK® accession numbers are reported in the text.

FIG. 4. Amino acid pairwise identity of LSV relative to other representative bunyaviruses. The amino acid identities are shown for the four LSV proteins (RdRp, G, N, and NSs). A sliding window of 50 base pairs (bp) was used. GENBANK® accession numbers are reported in the text.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The sequence listing is submitted as an ASCII text file, created on Mar. 12, 2013, 226 KB, which is incorporated by reference herein. In the accompanying sequence listing:

SEQ ID NO: 1 is the nucleotide sequence of the S segment of LSV.

SEQ ID NO: 2 is the nucleotide sequence of the M segment of LSV.

SEQ ID NO: 3 is the nucleotide sequence of the L segment of LSV.

SEQ ID NO: 4 is the amino acid sequence of the nucleocapsid protein (Np, S segment).

SEQ ID NO: 5 is the amino acid sequence of the nonstructural protein (NSs, S segment).

SEQ ID NO: 6 is the amino acid sequence of the glycoprotein precursor (M segment).

SEQ ID NO: 7 is the amino acid sequence of RdRP (L segment).

Nucleic acid and amino acid sequences are disclosed in GENBANK® Accession No. NC_021242.1, May 21, 2013; GENBANK® Accession No. KC589005.1, GENBANK® Accession No. May 18, 2013; GENBANK® Accession No. NC_021244.1, May 21, 2013, GENBANK® Accession No. NC_021243.1, May 21, 2013; GENBANK® Accession No. KC589007.1, May 18, 2013; and GENBANK® Accession No. KC589006.1, May 18, 2013, which are all incorporated by reference herein in their entirety.

### DETAILED DESCRIPTION

Disclosed herein is the LSV, which is a member of the genus Phlebovirus (family Bunyaviridae). In particular, disclosed herein are the complete nucleotide sequences of the LSV, including the sequences of the L, M and S segments, as well as the amino acid sequences of the proteins encoded by this virus, including the non-structural protein (NSs) and glycoprotein (G), nucleocapsid (N) and RNA-dependent RNA polymerase (RdRp) proteins. Antibodies are disclosed that specifically bind to a LSV polypeptide. In some embodiments, oligonucleotides that specifically hybridize with an LSV nucleic acid, including primers and probes, and antibodies that specifically bind a protein encoded by a LSV are provided. Also disclosed are diagnostic and detection assays using the Phlebovirus nucleic antibodies, proteins, probes, primers and nucleic acid molecules. Further provided are recombinant Phleboviruses, such as recombinant LSV encoding a reporter molecule and/or comprising an attenuating mutation. Methods for eliciting an immune response to LSV are also disclosed.

### Terms

Unless otherwise noted, technical terms are used according to conventional usage.

Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). In order to facilitate review of the various embodiments of the disclosure, the following explanations of specific terms are provided:

Adjuvant: A substance or vehicle that non-specifically enhances the immune response to an antigen. Adjuvants can include a suspension of minerals (alum, aluminum hydroxide, or phosphate) on which antigen is adsorbed; or water-in-oil emulsion in which antigen solution is emulsified in mineral oil (for example, Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants also include biological molecules, such as costimulatory molecules. Exemplary biological adjuvants include IL-2, RANTES, GM-CSF, TNF-α, IFN-γ, G-CSF, LFA-3, CD72, B7-1, B7-2, OX-40L and 4-1BBL.

Administer: To give, apply or bring the composition into contact with the subject. Administration can be accomplished by any of a number of routes, such as, for example, topical, oral, intranasal, subcutaneous, intramuscular, intraperitoneal, intravenous and intrathecal. A composition can be administered therapeutically or prophylactically. Prophylactic administration can occur prior to manifestation of symptoms characteristic of an infection.

Ambisense: Refers to a genome or genomic segments having both positive sense and negative sense portions. For example, the S segment of a Phlebovirus is ambisense, encoding nucleoprotein (NP) in the negative sense and the non-structural protein (NSs) in the positive sense.

Animal: Living multicellular vertebrate organisms, a category which includes, for example, mammals and birds.

Antibody: A polypeptide ligand comprising at least a light chain or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody.

Antibodies include intact immunoglobulins and the variants and portions of antibodies well known in the art, such as Fab fragments, Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W. H. Freeman & Co., New York, 1997.

Typically, a naturally occurring immunoglobulin has heavy (H) chains and light (L) chains interconnected by disulfide bonds. There are two types of light chain, lambda (λ) and kappa (k). There are five main heavy chain classes (or isotypes) which determine the functional activity of an antibody molecule: IgM, IgD, IgG, IgA and IgE.

Each heavy and light chain contains a constant region and a variable region (the regions are also known as “domains”). References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimeric antibody” has framework residues, or a constant domain, from one species, such as human, and includes the CDRs (which generally confer antigen binding) from another species, such as a murine antibody.

A “humanized” immunoglobulin is an immunoglobulin including a human framework region and one or more complementarity determining regions (CDRs) from a non-human (for example a mouse, rat, or synthetic) immunoglobulin. A humanized antibody can also include a human constant domain and a variable domain from a non-human antibody. The non-human immunoglobulin providing the CDRs is termed a “donor,” and the human immunoglobulin providing the framework is termed an “acceptor.” In some embodiments, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. Humanized immunoglobulins can be constructed by means of genetic engineering (see for example, U.S. Pat. No. 5,585,089).

A “human” antibody (also called a “fully human” antibody) is an antibody that includes human framework regions and all of the CDRs from a human immunoglobulin. In one example, the framework and the CDRs are from the same originating human heavy and/or light chain amino acid sequence. However, frameworks from one human antibody can be engineered to include CDRs from a different human antibody. All parts of a human immunoglobulin are substantially identical to corresponding parts of natural human immunoglobulin sequences.

Antigen: A compound, composition, or substance that can stimulate the production of antibodies or a T-cell response in an animal, including compositions that are injected or absorbed into an animal. An antigen reacts with the products of specific humoral or cellular immunity.

Anti-Genomic: As used herein, “anti-genomic” refers to a genomic segment of a Phlebovirus in the orientation opposite to the viral genome. For example, Phleboviruses are negative-sense RNA viruses. Thus, “anti-genomic” refers to the positive-sense orientation (or virus complementary sense), while “genomic” refers to the negative-sense orientation of a gene segment.

Attenuated: In the context of a live virus, the virus is attenuated if its ability to infect a cell or subject and/or its ability to produce disease is reduced (for example, eliminated) compared to a wild-type virus. Typically, an attenuated virus retains at least some capacity to elicit an immune response following administration to an immunocompetent subject. In some cases, an attenuated virus is capable of eliciting a protective immune response without causing any signs or symptoms of infection. In some embodiments, the ability of an attenuated virus to cause disease in a subject is reduced at least about 10%, at least about 25%, at least about 50%, at least about 75% or at least about 90% relative to wild-type virus. Accordingly, an “attenuating mutation” is a mutation in the viral genome and/or an encoded polypeptide that results in an attenuated virus.

Binding or Stable Binding: An oligonucleotide binds or stably binds to a target nucleic acid if a sufficient amount of the oligonucleotide forms base pairs or is hybridized to its target nucleic acid, to permit detection of that binding. Binding can be detected by either physical or functional properties of the target:oligonucleotide complex. Binding between a target and an oligonucleotide can be detected by any procedure known to one skilled in the art, including both functional or physical binding assays. Binding may be detected functionally by determining whether binding has an observable effect upon a biosynthetic process such as expression of a gene, DNA replication, transcription, translation and the like.

Physical methods of detecting the binding of complementary strands of DNA or RNA are well known in the art, and include such methods as DNase I or chemical footprinting, gel shift and affinity cleavage assays, Northern blotting, dot blotting and light absorption detection procedures. For example, a method which is widely used, because it is so simple and reliable, involves observing a change in light absorption of a solution containing an oligonucleotide (or an analog) and a target nucleic acid at 220 to 300 nm as the temperature is slowly increased. If the oligonucleotide or analog has bound to its target, there is a sudden increase in absorption at a characteristic temperature as the oligonucleotide (or analog) and target dissociate or melt.

The binding between an oligomer and its target nucleic acid is frequently characterized by the temperature (Tₘ) at which 50% of the oligomer is melted from its target. A higher (Tₘ) means a stronger or more stable complex relative to a complex with a lower (Tₘ).

Biological Sample: A sample obtained from a subject (such as a human or veterinary subject). Exemplary biological samples include fluid, cell and/or tissue samples. In some embodiments herein, the biological sample is a fluid sample. Fluid samples include, but are not limited to, serum, blood, plasma, urine, feces, saliva, cerebral spinal fluid (CSF) or other bodily fluid. Biological samples can also refer to cells or tissue samples, such as biopsy samples, tissue sections or isolated leukocytes.

Contacting: Placement in direct physical association; includes both in solid and liquid form. “Contacting” is often used interchangeably with “exposed.” In some cases, “contacting” includes transfecting, such as transfecting a nucleic acid molecule into a cell. In other examples, “contacting” refers to incubating a molecule (such as an antibody) with a biological sample.

Detecting: Determining the presence, using any method, of the virus or viral particles including viral peptides, inside cells, on cells, and/or in medium with which cells or the virus have come into contact. The methods are exemplified by, but not limited to, the observation of cytopathic effect, detection of viral protein, such as by immunofluorescence, ELISA, or Western blot hybridization, detection of viral nucleic acid sequence, such as by PCR, RT-PCR, Southern blots, and Northern blots, nucleic acid hybridization, nucleic acid arrays, and the like.

Expression Vector: A plasmid, a virus or another medium, known in the art, into which a nucleic acid sequence for encoding a desired protein can be inserted or introduced.

Fluorophore: A chemical compound, which when excited by exposure to a particular wavelength of light, emits light (i.e., fluoresces), for example at a different wavelength. In some embodiments herein, a probe is labeled with a fluorophore, such as at the 5′ end of the probe. Probes used for real-time PCR assays typically include a fluorophore and a quencher. Fluorophores suitable for use with real-time PCR assays, such as TaqMan™ PCR, include, but are not limited to, 6-carboxyfluorescein (FAM), tetrachlorofluorescein (TET), tetramethylrhodamine (TMR), hexachlorofluorescein (HEX), JOE, ROX, CAL FLUOR™, PULSAR™, QUASAR™, TEXAS RED™, CY™3 AND CY™5.

Heterologous: A heterologous sequence is a sequence that is not normally (i.e. in the wild-type sequence) found adjacent to a second sequence. In one embodiment, the sequence is from a different genetic source, such as a different virus or organism, than the second sequence.

Host Cell: A cell that is susceptible to transformation, transfection, transduction, conjugation, and the like with an exogenous nucleic acid construct or expression vector. Host cells can be from mammals, plants, bacteria, yeast, fungi, insects, animals, etc. A host cell can be from a human or a non-human primate.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence.

“Specifically hybridizable” and “specifically complementary” are terms that indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired, for example under physiological conditions in the case of in vivo assays or systems. Such binding is referred to as specific hybridization.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization buffer will determine the stringency of hybridization, though wash times also influence stringency. Calculations regarding hybridization conditions required for attaining particular degrees of stringency are discussed by Sambrook et al. (ed.), Molecular Cloning: A Laboratory Manual, 2^{nd} ed., vol. 1-3, Cold Spring Harbor Laboratory Press, Cold Spring Harbor, N. Y., 1989, chapters 9 and 11; and Ausubel et al. Short Protocols in Molecular Biology, 4^{th} ed., John Wiley & Sons, Inc., 1999.

For purposes of the present disclosure, “stringent conditions” encompass conditions under which hybridization will only occur if there is less than 25% mismatch between the hybridization molecule and the target sequence. “Stringent conditions” may be broken down into particular levels of stringency for more precise definition. Thus, as used herein, “moderate stringency” conditions are those under which molecules with more than 25% sequence mismatch will not hybridize; conditions of “medium stringency” are those under which molecules with more than 15% mismatch will not hybridize, and conditions of “high stringency” are those under which sequences with more than 10% mismatch will not hybridize. Conditions of “very high stringency” are those under which sequences with more than 6% mismatch will not hybridize.

“Specific hybridization” refers to the binding, duplexing, or hybridizing of a molecule only or substantially only to a particular nucleotide sequence when that sequence is present in a complex mixture (for example, total cellular DNA or RNA). Specific hybridization may also occur under conditions of varying stringency. Hybridization conditions resulting in particular degrees of stringency will vary depending upon the nature of the hybridization method and the composition and length of the hybridizing nucleic acid sequences. The following is an exemplary set of hybridization conditions and is not limiting:

Very High Stringency (Detects Sequences that Share at Least 90% Identity)

| | |
|---|---|
| Hybridization: | 5x SSC at 65° C. for 16 hours |
| Wash twice: | 2x SSC at room temperature (RT) for 15 minutes each |
| Wash twice: | 0.5x SSC at 65° C. for 20 minutes each |

High Stringency (Detects Sequences that Share at Least 80% Identity)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (Detects Sequences that Share at Least 60% Identity)

| | |
|---|---|
| Hybridization: | 6x SSC at RT to 55° C. for 16-20 hours |
| Wash at least twice: | 2x-3x SSC at RT to 55° C. for 20-30 minutes each |

Immune response: A response of a cell of the immune system, such as a B-cell, T-cell, macrophage or polymorphonucleocyte, to a stimulus such as an antigen. An immune response can include any cell of the body involved in a host defense response, including for example, an epithelial cell that secretes an interferon or a cytokine. An immune response includes, but is not limited to, an innate immune response or inflammation. As used herein, a protective immune response refers to an immune response that protects a subject from infection (prevents infection or prevents the development of disease associated with infection).

Immunogen: A compound, composition, or substance which is capable, under appropriate conditions, of stimulating an immune response, such as the production of antibodies or a T-cell response in an animal, such as to LSV. An immunogen includes compositions that are injected or absorbed into an animal. As used herein, an “immunogenic composition” is a composition comprising an immunogen.

Immunize: To render a subject protected from an infectious disease, such as by vaccination.

Isolated: An “isolated” biological component (such as a nucleic acid, protein or virus) has been substantially separated or purified away from other biological components (such as cell debris, or other proteins or nucleic acids). Biological components that have been “isolated” include those components purified by standard purification methods. The term also embraces recombinant nucleic acids, proteins or viruses, as well as chemically synthesized nucleic acids or peptides. An isolated composition can be at least 90%, 91%, 92%, 93%, 94%, 95%, 96%, 97%, 98%, 99% or 100% pure.

Label: A detectable moiety or any atom, molecule or a portion thereof, the presence, absence or level of which is directly or indirectly monitorable. A variety of detectable moieties are well known to those skilled in the art, and can be any material detectable by spectroscopic, photochemical, biochemical, immunochemical, electrical, optical or chemical means. Such detectable labels can include, but are not limited to, magnetic beads, fluorescent dyes, radiolabels, enzymes, and colorimetric labels such as colloidal gold or colored glass or plastic beads.

Mammal: This term includes both human and non-human mammals. Similarly, the term “subject” includes both human and veterinary subjects.

Nucleic acid: Deoxyribonucleotides, ribonucleotides, and polymers thereof, in either single-stranded or double-stranded form. This term includes complements of single stranded nucleotides and cDNAs. Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleotide sequence can encompass “splice variants,” which as the name suggests, are products of alternative splicing of a gene. After transcription, an initial nucleic acid transcript can be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. A polynucleotide is generally a linear nucleotide sequence, including sequences of greater than 100 nucleotide bases in length.

Oligonucleotide: A short nucleic acid polymer. Oligonucleotides are generally less than 100 nucleotides in length. In some embodiments herein, the oligonucleotide is 8-100, 10-50, 12-40, 16-30 or 18-24 nucleotides in length. In particular examples, the oligonucleotide is 12, 13, 14, 15, 16, 17, 18, 19, 20, 21, 22, 23, 24, 25, 26, 27, 28, 29 or 30 nucleotides in length.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

ORF (open reading frame): A series of nucleotide triplets (codons) coding for amino acids without any termination codons. These sequences are usually translatable into a peptide.

Pharmaceutically acceptable carrier: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds or molecules, such as one or more recombinant Phleboviruses, and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Phlebovirus: One of five genera of the Bunyaviridae family. Phleboviruses are enveloped spherical viruses with icosahedral symmetry. The genome of Phleboviruses consists of three single-stranded RNA genome segments—small (S), medium (M) and large (L). The M and L segments are negative sense RNA strands, while the S segment is ambisense RNA. The S segment encodes the non-structural protein (NSs) in the positive sense orientation and the nucleoprotein (NP) in the negative sense orientation. The M segment encodes the glycoprotein precursor that is cleaved by host proteases into two structural domains—Gn and Gc. The L segment encodes the L protein, which functions as the RNA dependent RNA polymerase in primary and secondary transcription to generate mRNA and replicative intermediates, respectively. Phleboviruses have a worldwide distribution and are transmitted by a wide variety of arthropods, including sandflies, mosquitoes and ticks. Several Phleboviruses have been linked to human disease, in some cases causing febrile illness, fever, hepatitis, meningitis, encephalitis or hemorrhagic syndrome. LSV is a Phlebovirus.

Polypeptide: A polymer in which the monomers are amino acid residues which are joined together through amide bonds. When the amino acids are alpha-amino acids, either the L-optical isomer or the D-optical isomer can be used. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced. The term “residue” or “amino acid residue” includes reference to an amino acid that is incorporated into a protein, polypeptide, or peptide.

Conservative amino acid substitutions are those substitutions that, when made, least interfere with the properties of the original protein, that is, the structure and especially the function of the protein is conserved and not significantly changed by such substitutions. Examples of conservative substitutions are shown below.

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Probes and primers: A probe comprises an isolated nucleic acid molecule attached to a detectable label or other reporter molecule. Typical labels include radioactive isotopes, enzyme substrates, co-factors, ligands, chemiluminescent or fluorescent agents, haptens, and enzymes. Methods for labeling and guidance in the choice of labels appropriate for various purposes are discussed, for example, in Sambrook et al. (ed.), Molecular Cloning: A Laboratory Manual, 2^{nd} ed., vol. 1-3, Cold Spring Harbor Laboratory Press, Cold Spring Harbor, N. Y., 1989 and Ausubel et al. Short Protocols in Molecular Biology, 4^{th} ed., John Wiley & Sons, Inc., 1999.

Primers are short nucleic acid molecules, for instance DNA oligonucleotides 10 nucleotides or more in length, for example that hybridize to contiguous complementary nucleotides or a sequence to be amplified. Longer DNA oligonucleotides may be about 12, 15, 18, 20, 25, 30, or 50 nucleotides or more in length. Primers can be annealed to a complementary target DNA strand by nucleic acid hybridization to form a hybrid between the primer and the target DNA strand, and then the primer extended along the target DNA strand by a DNA polymerase enzyme. Primer pairs can be used for amplification of a nucleic acid sequence, for example, by the polymerase chain reaction (PCR) or other nucleic-acid amplification methods known in the art. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in WO 90/01069; ligase chain reaction amplification; gap filling ligase chain reaction amplification, as disclosed in U.S. Pat. No. 5,427,930; and NASBA™ RNA transcription-free amplification, as disclosed in U.S. Pat. No. 6,025,134.

Methods for preparing and using nucleic acid probes and primers are described, for example, in Sambrook et al. (ed.), Molecular Cloning: A Laboratory Manual, 2^{nd} ed., vol. 1-3, Cold Spring Harbor Laboratory Press, Cold Spring Harbor, N. Y., 1989; Ausubel et al. Short Protocols in Molecular Biology, 4^{th} ed., John Wiley & Sons, Inc., 1999; and Innis et al. PCR Protocols, A Guide to Methods and Applications, Academic Press, Inc., San Diego, Calif., 1990. Amplification primer pairs can be derived from a known sequence, for example, by using computer programs intended for that purpose such as Primer (Version 0.5, © 1991, Whitehead Institute for Biomedical Research, Cambridge, Mass.).

Quencher: A substance that absorbs excitation energy from a fluorophore when in close proximity. Probes used for real-time PCR assays, such as TAQMAN™ PCR, typically include a fluorophore and a quencher. Quenchers suitable for use with real-time PCR assays include, but are not limited to, ZEN™, IOWA BLAck™ FQ, tetramethylrhodamine (TAMRA), black hole quencher (BHQ)1, BHQ2, BHQ3 and 4-(4′-dimethylaminophenylazo)benzoic acid (DABCYL). In some examples, a probe contains two quenchers.

Recombinant: A recombinant nucleic acid, protein or virus is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, for example, by genetic engineering techniques. In some examples, the recombinant Phleboviruses comprise one or more deletions in a viral virulence factor, such as NSs. In other examples, the recombinant viruses include a heterologous gene, such as a reporter gene.

Reporter gene: A reporter gene is a gene operably linked to another gene or nucleic acid sequence of interest (such as a promoter sequence). Reporter genes are used to determine whether the gene or nucleic acid of interest is expressed in a cell or has been activated in a cell. Reporter genes typically have easily identifiable characteristics, such as fluorescence, or easily assayed products, such as an enzyme. Reporter genes can also confer antibiotic resistance to a host cell or tissue. Reporter genes include, for example, labels such as green fluorescent protein (GFP or eGFP) or other fluorescence genes, luciferase, β-galactosidase and alkaline phosphatase.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a given gene or protein will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237-244, 1988; Higgins and Sharp, CABIOS 5:151-153, 1989; Corpet et al., Nucleic Acids Research 16:10881-10890, 1988; and Pearson and Lipman, Proc. Natl. Acad. Sci. U.S.A. 85:2444, 1988. Altschul et al., Nature Genet. 6:119-129, 1994.

The NCBI Basic Local Alignment Search Tool (BLAST™) (Altschul et al., J. Mol. Biol. 215:403-410, 1990) is available from several sources, including the National Center for Biotechnology Information (NCBI, Bethesda, Md.) and on the Internet, for use in connection with the sequence analysis programs blastp, blastn, blastx, tblastn and tblastx.

In some embodiments herein, provided are nucleotide or amino acid sequences at least 80%, at least 85%, at least 90%, at least 95%, at least 96%, at least 97%, at least 98% or at least 99% identical to any one of SEQ ID NOs: 1-10.

Subject: Living multi-cellular vertebrate organisms, a category that includes both human and non-human mammals.

Therapeutically effective amount: A quantity of a specified agent sufficient to achieve a desired effect in a subject being treated with that agent. For example, this may be the amount of a recombinant Phlebovirus, such as the Lone Star Virus, useful for eliciting an immune response in a subject and/or for preventing infection by Phlebovirus. Ideally, in the context of the present disclosure, a therapeutically effective amount of a recombinant Phlebovirus is an amount sufficient to increase resistance to, prevent, ameliorate, and/or treat infection caused by Phlebovirus in a subject without causing a substantial cytotoxic effect in the subject. The effective amount of a recombinant Phlebovirus useful for increasing resistance to, preventing, ameliorating, and/or treating infection in a subject will be dependent on, for example, the subject being treated, the manner of administration of the therapeutic composition and other factors.

Vaccine: A preparation of immunogenic material capable of stimulating an immune response, administered for the prevention, amelioration, or treatment of infectious or other types of disease. The immunogenic material may include attenuated or killed microorganisms (such as attenuated viruses), antigenic proteins, peptides or DNA encoding an antigenic protein or peptide.

Vaccines may elicit both prophylactic (preventative) and therapeutic responses. Methods of administration vary according to the vaccine, but may include inoculation, ingestion, inhalation or other forms of administration. Inoculations can be delivered by any of a number of routes, including parenteral, such as intravenous, subcutaneous or intramuscular. Vaccines may be administered with an adjuvant to boost the immune response.

Vector: A nucleic acid molecule as introduced into a host cell, thereby producing a transformed host cell. A vector may include nucleic acid sequences that permit it to replicate in a host cell, such as an origin of replication (DNA sequences that participate in initiating DNA synthesis). A vector may also include one or more selectable marker genes and other genetic elements known in the art.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. All GENBANK® Accession numbers are incorporated by reference herein as they appear in the database on Apr. 19, 2013. In case of conflict, the present specification, including explanations of terms, will control. In addition, the materials, methods, and examples are illustrative only and not intended to be limiting.

### III. Overview of Several Embodiments

Disclosed herein is the discovery of a new member of the genus Phlebovirus (family Bunyaviridae) called Lone Star Virus (LSV). In some embodiments, infection with a Phlebovirus disclosed herein is associated with recent tick bite.

In particular, provided herein are the complete nucleotide sequences of all three genome segments of LSV, as well as the amino acid sequences of the proteins encoded by each isolate, including the NP, GP, NSs and polymerase (L) proteins. Antibodies, such as monoclonal antibodies, antigen-binding fragments thereof, and chimeric forms thereof (such as humanized antibodies) are also provided. Oligonucleotides, such as primers and probes, that specifically hybridize with the LSV nucleic acid sequences, and antibodies specific for the encoded proteins are further provided. Also provided are diagnostic and detection assays using the LSV nucleic acid molecules, proteins, probes, primers and antibodies. In addition, provided are recombinant Phleboviruses, such as recombinant viruses encoding a reporter molecule and/or comprising an attenuating mutation, and their use for eliciting an immune response in a subject.

### Lone Star Virus Nucleic Acid Molecules and Polypeptides

Nucleic acid molecules are disclosed herein, such as an S segment, an M segment and an L segment at least 80% identical to the S segment, M segment or L segment, respectively, of a Lone Star Virus (LSV) isolate. In some embodiments, the nucleotide sequence of the S segment is at least 80%, at least 85%, at least 90%, at least 95%, at least 96%, at least 97%, at least 98% or at least 99% identical to SEQ ID NO: 1. In other embodiments, the nucleotide sequence of the M segment is at least 80%, at least 85%, at least 90%, at least 95%, at least 96%, at least 97%, at least 98% or at least 99% identical to SEQ ID NO: 2. In additional embodiments, the nucleotide sequence of the L segment is at least 80%, at least 85%, at least 90%, at least 95%, at least 96%, at least 97%, at least 98% or at least 99% identical to SEQ ID NO: 3.

In some non-limiting examples, the nucleotide sequence of the S segment comprises or consists of SEQ ID NO: 1; the nucleotide sequence of the M segment comprises or consists of SEQ ID NO: 2; the nucleotide sequence of the L segment comprises or consists of SEQ ID NO: 3; or any combination thereof. In particular non-limiting examples, the S segment comprises or consists of SEQ ID NO: 1, the M segment comprises or consists of SEQ ID NO: 2 and the L segment comprises or consists of SEQ ID NO: 3.

In additional embodiments, nucleic acid molecules are provided that are at least 80%, at least 85%, at least 90%, at least 95%, at least 96%, at least 97%, at least 98% or at least 99% identical to SEQ ID NO: 1, SEQ ID NO: 2; or SEQ ID NO: 3, or the complement thereof. In more embodiments, nucleic acid molecules are provided that are at least 80%, at least 85%, at least 90%, at least 95%, at least 96%, at least 97%, at least 98% or at least 99% identical to SEQ ID NO: 1, SEQ ID NO: 2; or SEQ ID NO: 3. Nucleic acid molecules comprising or consisting of SEQ ID NOs: 1, 2 and/or 3 are also provided. Any of these nucleic acid molecule can be RNA or cDNA.

In additional embodiments, nucleic acid molecules are provided that include a nucleic acid sequence at least 80%, 85%, 90%, 91%, 92%, 93%, 94%, 95%, 96%, 97%, 98%, 99%, or 100% identical to an open reading frame of one of SEQ ID NO: 1, the complement thereof, SEQ ID NO: 2 or SEQ ID NO: 3. In some examples, the nucleic acid molecule includes, or consists of, an open reading frame of the nucleic acid sequence set forth as SEQ ID NO: 1, the complement thereof, SEQ ID NO: 2 or SEQ ID NO: 3. In specific examples, the nucleic acid molecule includes or consists of, the nucleic acid sequence set forth as SEQ ID NO: 1, the complement thereof, SEQ ID NO: 2 or SEQ ID NO: 3. Isolated nucleic acid molecules are provided that comprise, or consist of, a nucleic acid molecule encoding one or more of SEQ ID NOs: 4-7. Any of these nucleic acid molecules can be RNA or cDNA.

Further provided are isolated polypeptides encoded by an open reading frame of an LSV nucleic acid molecule. In some embodiments, the amino acid sequence of the LSV-encoded polypeptide is at least 80%, at least 85%, at least 90%, at least 95%, at least 96%, at least 97%, at least 98% or at least 99% identical to polypeptide encoded by an open reading frame of SEQ ID NO: 1, the complement thereof, SEQ ID NO: 2, or SEQ ID NO: 3. In particular examples, the amino acid sequence of the LSV polypeptide comprises or consists of an amino acid sequence at least 80%, at least 85%, at least 90%, at least 95%, at least 96%, at least 97%, at least 98% or at least 99% identical to one of SEQ ID NOs: 4-7. In other examples, the LSV polypeptide comprises or consists of the amino acid sequence set forth as one of SEQ ID NOs: 4-7. In other examples, the LSV polypeptide comprises or consists of an amino acid sequence with at most 1, 2, 3, 4, or 5 conservative amino acid substitutions in the amino acid sequence set forth as one of SEQ ID NOs: 4-7.

Vectors comprising any of the nucleic acid molecules disclosed herein, or encoding the proteins and peptides disclosed herein, are provided by the present disclosure, and can be used to transform cells. The vector can be any suitable vector, such as a plasmid vector or a viral vector. In some embodiments, the vector comprises a promoter, an origin of replication and/or a selectable marker. The vector can also encode a reporter. In some examples, the nucleic acid molecule of the vector is operably linked to a promoter. Exemplary promoters include viral promoters, such as cytomegalovirus immediate early gene promoter (“CMV”), herpes simplex virus thymidine kinase (“tk”), SV40 early transcription unit, polyoma, retroviruses, papilloma virus, hepatitis B virus, and human and simian immunodeficiency viruses. Other promoters are isolated from mammalian genes, including the immunoglobulin heavy chain, immunoglobulin light chain, T-cell receptor, HLA DQ α and DQ β, β-interferon, interleukin-2, interleukin-2 receptor, MHC class II, HLA-DRα, β-actin, muscle creatine kinase, prealbumin (transthyretin), elastase I, metallothionein, collagenase, albumin, fetoprotein, β-globin, c-fos, c-HA-ras, insulin, neural cell adhesion molecule (NCAM), α1-antitrypsin, H2B (TH2B) histone, type I collagen, glucose-regulated proteins (GRP94 and GRP78), rat growth hormone, human serum amyloid A (SAA), troponin I (TNI), platelet-derived growth factor, and dystrophin, dendritic cell-specific promoters, such as CD11c, macrophage-specific promoters, such as CD68, Langerhans cell-specific promoters, such as Langerin, and promoters specific for keratinocytes, and epithelial cells of the skin and lung.

In some embodiments, the promoter is inducible. An inducible promoter is a promoter which is inactive or exhibits low activity except in the presence of an inducer substance. Examples of inducible promoters include, but are not limited to, MT II, MMTV, collagenase, stromelysin, SV40, murine MX gene, α-2-macroglobulin, MHC class I gene h-2kb, HSP70, proliferin, tumor necrosis factor, or thyroid stimulating hormone gene promoter. In other embodiments, the promoter is a constitutive promoter that results in high levels of transcription upon introduction into a host cell in the absence of additional factors. Optionally, the transcription control sequences include one or more enhancer elements, which are binding recognition sites for one or more transcription factors that increase transcription above that observed for the minimal promoter alone.

It may be desirable to include a polyadenylation signal to effect proper termination and polyadenylation of the gene transcript. Exemplary polyadenylation signals have been isolated from bovine growth hormone, SV40 and the herpes simplex virus thymidine kinase genes. Any of these or other polyadenylation signals can be utilized in the context of the adenovirus vectors described herein.

The vector can be, for example, a viral vector. A number of viral vectors have been constructed, including polyoma, SV40 (Madzak et al., 1992, J. Gen. Virol., 73:15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Nad. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell Biol., 5:431-437; Sorge et al., 1984, Mol. Cell Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.).

Also provided are host cells including these vectors. The host cell can be a eukaryotic cell or a prokaryotic cell. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, Jakoby and Pastan (eds), 1979, Cell Culture. Methods in Enzymology, volume 58, Academic Press, Inc., Harcourt Brace Jovanovich, N.Y.). Examples of commonly used mammalian host cell lines are VERO and HeLa cells, CHO cells, and WI38, BHK, and COS cell lines, although cell lines may be used, such as cells designed to provide higher expression desirable glycosylation patterns, or other features. Techniques for the transformation of yeast cells, such as polyethylene glycol transformation, protoplast transformation and gene guns are also known in the art (see Gietz and Woods Methods in Enzymology 350: 87-96, 2002).

Transformation of a host cell with recombinant DNA can be carried out by conventional techniques as are well known to those skilled in the art. Where the host is prokaryotic, such as, but not limited to, E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation. When the host is a eukaryote, such methods of transfection of DNA as calcium phosphate coprecipitates, conventional mechanical procedures such as microinjection, electroporation, insertion of a plasmid encased in liposomes, or virus vectors can be used.

A number of procedures can be employed when recombinant protein is being purified, such as from a host cell. For example, proteins having established molecular adhesion properties can be reversible fused to the protein. With the appropriate ligand or substrate, a specific protein can be selectively adsorbed to a purification column and then freed from the column in a relatively pure form. The fused protein is then removed by enzymatic activity. Finally, protein could be purified using immunoaffinity columns. Recombinant protein can be purified from any suitable source, include yeast, insect, bacterial, and mammalian cells.

Recombinant proteins can be expressed from recombinant nucleic acids, such as from plasmids, and purified by transformed bacteria in large amounts, typically after promoter induction; but expression can be constitutive. Promoter induction with IPTG is one example of an inducible promoter system. Bacteria are grown according to standard procedures in the art. Fresh or frozen bacteria cells are used for isolation of protein.

Proteins expressed in bacteria can form insoluble aggregates (“inclusion bodies”). Several protocols are suitable for purification of protein inclusion bodies. For example, purification of inclusion bodies typically involves the extraction, separation and/or purification of inclusion bodies by disruption of bacterial cells, e.g., by incubation in a buffer of 50 mM TRIS/HCL pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a homignizer, such as Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies can be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation can occur upon removal (by dialysis, for example) or dilution of the denaturant, allowing re-formation of immunologically and/or biologically active protein. Other suitable buffers are known to those skilled in the art. Human proteins are separated from other bacterial proteins by standard separation techniques, e.g., with Ni-NTA agarose resin.

Alternatively, it is possible to purify recombinant protein from bacteria periplasm. After lysis of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO4 and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

Solubility fractionation can be used as a standard protein separation technique for purifying proteins. As an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

The molecular weight of the protein can be used to isolate it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

The protein can also be separated from other proteins on the basis of its size, net surface charge, hydrophobicity, and affinity for ligands or substrates using column chromatography. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

Also provided are oligonucleotides that specifically hybridize with a LSV nucleic acid molecule. Oligonucleotides are generally less than 100 nucleotides in length. In some embodiments, the oligonucleotide is less than 80, less than 60, less than 40 or less than 30 nucleotides in length. In other embodiments, the oligonucleotide is 8-100, 10-50, 12-40, 16-30 or 18-24 nucleotides in length. In particular examples, the oligonucleotide is 12, 13, 14, 15, 16, 17, 18, 19, 20, 21, 22, 23, 24, 25, 26, 27, 28, 29 or 30 nucleotides in length. In one non-limiting example, the oligonucleotide is 12 to 40 nucleotides in length. In another non-limiting example, the oligonucleotide is 18 to 24 nucleotides in length. In some embodiments, the oligonucleotide is at least 85%, at least 90%, at least 95%, at least 96%, at least 97%, at least 98% or at least 99% identical to consecutive nucleotides of SEQ ID NO: 1, 2 or 3. In additional embodiments, the oligonucleotide is at least 85%, at least 90%, at least 95%, at least 96%, at least 97%, at least 98% or at least 99% identical to consecutive nucleotides of the complement of SEQ ID NO: 1, 2 or 3. In some examples, the nucleotide sequence of the oligonucleotide comprises or consists of nucleotides of SEQ ID NO: 1, 2, or 3. In additional examples, the nucleotide sequence of the oligonucleotide comprises or consists of nucleotides of the complement of SEQ ID NO: 1, 2, or 3.

In some embodiments, the oligonucleotide comprises a fluorophore. Numerous fluorophores are known in the art and an appropriate fluorophore can be selected by a skilled artisan based on the intended use of the oligonucleotide. As one example, for real-time PCR assays, such as TAQMAN™ PCR, exemplary fluorophores include, but are not limited to, FAM, TET, TMR, HEX, JOE, ROX, CAL FLUOR™, PULSAR™, QUASAR™, TEXAS RED™, CY™3 and CY™5.

In some embodiments, the oligonucleotide comprises a quencher. In some instances, the oligonucleotide comprises more than one quencher, such as two quenchers. A suitable quencher (or quenchers) can be selected by one of skill in the art depending on the intended purpose of the oligonucleotide. As one example, for real-time PCR assays, such as TAQMAN™ PCR, exemplary quenchers include, but are not limited to, ZEN™, IOWA BLACK™ FQ, TAMRA, BHQ1, BHQ2, BHQ3 and DABCYL™. In some examples, the oligonucleotide includes two quenchers.

In one non-limiting example, the oligonucleotide, such as when the oligonucleotide will be used as a probe, comprises a fluorophore and a quencher. In another non-limiting example, the oligonucleotide comprises a fluorophore and two quenchers. The oligonucleotide can be a probe or a primer.

### Phleboviruses

The Lone Star Virus is disclosed herein. In some embodiments, the genome of the LSV comprises an S segment having a nucleotide sequence at least 85%, at least 90%, at least 95%, at least 96%, at least 97%, at least 98% or at least 99% identical to SEQ ID NO: 1; an M segment having a nucleotide sequence at least 85%, at least 90%, at least 95%, at least 96%, at least 97%, at least 98% or at least 99% identical to SEQ ID NO: 2; or an L segment having a nucleotide sequence at least 85%, at least 90%, at least 95%, at least 96%, at least 97%, at least 98% or at least 99% identical to SEQ ID NO: 3. In other embodiments, the genome of the recombinant LSV comprises an S segment having a nucleotide sequence at least 85%, at least 90%, at least 95%, at least 96%, at least 97%, at least 98% or at least 99% identical to SEQ ID NO: 1; an M segment having a nucleotide sequence at least 85%, at least 90%, at least 95%, at least 96%, at least 97%, at least 98% or at least 99% identical to SEQ ID NO: 2; and an L segment having a nucleotide sequence at least 85%, at least 90%, at least 95%, at least 96%, at least 97%, at least 98% or at least 99% identical to SEQ ID NO: 3. In other non-limiting examples, (a) the nucleotide sequence of the S segment is at least 80%, at least 85%, at least 90%, at least 95%, at least 96%, at least 97%, at least 98% or at least 99% identical to SEQ ID NO: 1; (b) the nucleotide sequence of the M segment is at least 80%, at least 85%, at least 90%, at least 95%, at least 96%, at least 97%, at least 98% or at least 99% identical to SEQ ID NO: 2; and (c) the nucleotide sequence of the L segment is at least 80%, at least 85%, at least 90%, at least 95%, at least 96%, at least 97%, at least 98% or at least 99% identical to SEQ ID NO: 3, respectively.

In a specific non-limiting example, the genome of the recombinant Lone Star Virus includes an S segment at least 80% identical to SEQ ID NO: 1, an M segment at least 80% identical to SEQ ID NO: 2, and an L segment at least 80% identical to SEQ ID NO: 3. In another specific non-limiting example, the genome of the recombinant Lone Star Virus includes an S segment at least 90% identical to SEQ ID NO: 1, an M segment at least 90% identical to SEQ ID NO: 2, and an L segment at least 90% identical to SEQ ID NO: 3. In an additional specific non-limiting example, the genome of the recombinant Lone Star Virus includes an S segment at least 95% identical to SEQ ID NO: 1, an M segment at least 95% identical to SEQ ID NO: 2, and an L segment at least 95% identical to SEQ ID NO: 3.

In some embodiments, the genome of an LSV can include an S segment having a nucleotide sequence comprising SEQ ID NO: 1, an M segment comprising a nucleic acid sequence comprising SEQ ID NO: 2, and an L segment comprising a nucleic acid sequence comprising SEQ ID NO: 3. In additional embodiments, the genome of an LSV can include an S segment having a nucleotide sequence consisting of SEQ ID NO: 1, an M segment comprising a nucleic acid sequence consisting of SEQ ID NO: 2, and an L segment comprising a nucleic acid sequence consisting of SEQ ID NO: 3.

In some examples, the recombinant LSV comprises a deletion, such as a deletion of the NSs open reading frame (ORF). In particular examples, the deleted ORF is replaced with a reporter gene, such as a gene encoding a fluorescent protein or an antibiotic resistance gene.

In some examples, the recombinant LSV comprises at least one attenuating mutation. The attenuating mutation can be any insertion, deletion or substitution that results in a decrease in virus infectivity or virus-induced disease. The attenuating mutation can be in any of the gene segments and/or viral proteins. In some examples, the attenuating mutation results in an alteration or deletion of a virulence protein, such as NSs.

Recombinant LSV can be generated, for example, using a reverse genetics system. Reverse genetics systems for Phleboviruses are known in the art and are described in PCT Publication No. WO 2009/082647 and U.S. Pat. No. 8,084,248.

### Anti-LSV Antibodies

Provided herein are isolated antibodies, or antigen-binding fragments thereof, that specifically bind to LSV isolates and/or the LSV polypeptides disclosed herein. In some embodiments, the antibody is a polyclonal antibody. In other embodiments, the antibody is a monoclonal antibody.

In some embodiments, the antibody or antigen-binding fragment thereof specifically binds an epitope of a Phlebovirus virion, specifically an LSV virion, and can thus detect virus particles. In some embodiments, the antibody or antigen-binding fragment thereof specifically binds a linear epitope, or conformational epitope, or both, of a LSV polypeptide.

In some embodiments, the antibody or antigen-binding fragment thereof specifically binds to a NSs, NP, GP or L protein of LSV. In particular examples, the antibody or antigen-binding fragment thereof specifically binds a polypeptide that is at least 80%, at least 85%, at least 90%, at least 95%, at least 96%, at least 97%, at least 98% or at least 99% identical to one of SEQ ID NOs: 4-7. In other particular examples, the antibody or antigen-binding fragment thereof specifically binds a polypeptide with the amino acid sequence comprising or consisting of one of SEQ ID NOs: 4-7. The antibody can specifically bind an antigenic fragment of one of SEQ ID NOs: 4-7.

In some embodiments, the antigen-binding fragment is an Fab, Fab′, F(ab)′₂ scFv or dsFv. In some embodiments, the antibodies are mouse, rat or rabbit antibodies. In other embodiments, the antibodies are humanized antibodies or fully human antibodies. In other embodiments, the antibodies are chimeric antibodies.

For preparation of antibodies, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)).

Methods of production of polyclonal antibodies are known to those of skill in the art. In some embodiments, an inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the beta subunits. When appropriately high titers of antibody to the immunogen are obtained, blood is collected from the animal and antisera are prepared. Further fractionation of the antisera to enrich for antibodies reactive to the protein can be done if desired (see, Harlow & Lane, supra).

Monoclonal antibodies can be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells can be enhanced by various techniques, including injection into the peritoneal cavity of a vertebrate host. Alternatively, one can isolate DNA sequences which encode a monoclonal antibody or a binding fragment thereof by screening a DNA library from human B cells according to the general protocol outlined by Huse, et al., Science 246:1275-1281 (1989).

Phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-LSV proteins and nucleic acids, using a competitive binding immunoassay. Specific polyclonal antisera and monoclonal antibodies will usually bind with a Kd of at least about 0.1 mM, more usually at least about 1 uM, such as at least about 0.1 uM or better, for example, 0.01 uM or better. Antibodies specific only for a particular LSV protein can also be made by subtracting out other cross-reacting proteins. In this manner, antibodies that bind only to the protein of choice can be obtained.

Chimeric are also provided, wherein (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. In specific non-limiting examples, the antibody can be a chimeric antibody that includes the complementarity determining regions (CDRs) from one antibody that specifically binds the LSV protein, and at least one framework region from a different antibody.

Humanized or primatized antibodies can be used. Generally, a humanized antibody has one or more amino acid residues introduced into it from a source which is non-human. These non-human amino acid residues are often referred to as import residues, which are typically taken from an import variable domain. Methods for humanizing or primatizing non-human antibodies are well known in the art. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues, or all of the CDR residues and possibly some FR residues are substituted by residues from analogous sites in non-human, such as rodent antibodies. Humanized antibodies can also be antibodies wherein human framework regions are utilized, but the CDRs are from a non-human antibody.

Generally, the antibodies, and antigen-binding fragments specifically bind LSV. These LSV-specific antibodies can be used, for example, in assays for the detection of a LSV or LSV polypeptide in a biological sample. Exemplary immunodetection assays include the enzyme-linked immunosorbent assay (ELISA), Western blot, radioimmunoassay (RIA) and immunohistochemistry (IHC) assay. Methods of performing such assays are well known in the art and are briefly disclosed below.

### Methods for the Detection of Phlebovirus

The isolation and sequencing of LSV disclosed herein has enabled the development of a series of assays that can be used for the detection of LSV in a biological sample and/or the diagnosis of a LSV infection in a subject.

In some embodiments of the detection and diagnosis assays, the method further includes the step of obtaining a biological sample from a subject. In some examples, the sample is obtained directly from the subject and used in one of the above-described assays. In other examples, the sample is obtained indirectly without directly removing the biological sample from the subject. In some cases where the sample is obtained indirectly, the sample is obtained from, for example, a clinician or laboratory personnel.

In some embodiments, the biological sample is a cell or tissue sample, such as a biopsy sample, bone marrow aspirate or isolated cells. In other embodiments, the biological sample is a bodily fluid sample. In some examples, the bodily fluid sample comprises serum, blood, plasma, urine, feces, saliva or cerebral spinal fluid.

Provided herein is a method for detecting a LSV or LSV polypeptide in a biological sample using a LSV-specific antibody. In some embodiments, the method includes contacting the biological sample with a LSV-specific antibody, or antigen-binding fragment thereof; and detecting binding of the antibody or antigen-binding fragment to the biological sample. Binding of the antibody or antigen-binding fragment to the biological sample indicates the presence of the LSV or the LSV polypeptide in the biological sample.

In some embodiments of the detection method, the antibody or antigen-binding fragment thereof specifically binds to a NSs, NP, GP or L protein of LSV. In particular examples, the antibody or antigen-binding fragment thereof specifically binds a polypeptide that is at least 80%, at least 85%, at least 90%, at least 95%, at least 96%, at least 97%, at least 98% or at least 99% identical to an open reading frame of SEQ ID NOs: 1, 2, or 3. In other embodiments, the antibody or antigen-binding fragment thereof specifically binds a polypeptide that is at least 80%, at least 85%, at least 90%, at least 95%, at least 96%, at least 97%, at least 98% or at least 99% identical to one of SEQ ID NOs: 4-7. In other particular examples, the antibody or antigen-binding fragment thereof specifically binds a polypeptide with an amino acid sequence comprising or consisting of one of SEQ ID NOs: 4-7.

Also provided is a method for detecting LSV-specific antibodies in a biological sample using the LSV polypeptides disclosed herein. In some embodiments, the method includes contacting the biological sample with a LSV-specific polypeptide; and detecting binding of the polypeptide to the biological sample. Binding of the polypeptide to the biological sample indicates the presence of the LSV-specific antibodies in a biological sample.

In some embodiments of the detection method, the LSV polypeptide is a NSs, NP, GP or L protein. In particular examples, the amino acid sequence of the Phlebovirus polypeptide is at least 80%, at least 85%, at least 90%, at least 95%, at least 96%, at least 97%, at least 98% or at least 99% identical to one of SEQ ID NOs: 4-7. In other examples, the amino acid sequence of the Phlebovirus polypeptide comprises or consists of one of SEQ ID NOs: 4-7. The method detects one or more of these proteins in a biological sample from a subject of interest.

Detection assays based on binding of a polypeptide to an antibody are well known in the art and include, for example, ELISA, Western blot, fluorescence activated cell sorting (FACS), radioimmunoassay and immunohistochemistry. As is well known to one of skill in the art, in some cases the detection assay further includes the step of contacting an antigen-antibody complex with a detection reagent, such as a labeled secondary antibody (e.g., an anti-isotype antibody, such as an anti-IgG antibody), or in the case of a sandwich ELISA, a second antibody that recognizes the same antigen as the first antibody and is labeled for detection. Secondary antibodies can also be conjugated to magnetic beads to allow for magnetic sorting. In other cases, the primary antibody is directly labeled. Directly labeled antibodies can be used for a variety of detection assays, such as FACS.

Noncompetitive immunoassays are assays in which antigen is directly detected and, in some instances the amount of antigen directly measured. Enzyme mediated immunoassays such as immunofluorescence assays (IFA), enzyme linked immunosorbent assays (ELISA), immunoblotting (western), and capture assays can be readily adapted to accomplish the noncompetitive detection of the LSV proteins.

An ELISA method effective for the detection of the LSV can, for example, be as follows: (1) bind an antibody or antigen to a substrate; (2) contact the bound receptor with a fluid or tissue sample containing the virus, a viral antigen, or antibodies to the virus; (3) contact the above with an antibody bound to a detectable moiety (e.g., horseradish peroxidase enzyme or alkaline phosphatase enzyme); (4) contact the above with the substrate for the enzyme; (5) contact the above with a color reagent; (6) observe color change. The above method can be readily modified to detect presence of an anti-LSV antibody in the sample or a specific LSV polypeptide as well as the virus.

Western blot (immunoblot) analysis can be used to detect and quantify the presence of LSV in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the LSV polypeptide. The anti-LSV polypeptide antibodies specifically bind to the antigen on the solid support. These antibodies can be directly labeled or alternatively can be subsequently detected using labeled antibodies (e.g., labeled sheep anti-mouse antibodies) that specifically bind to the anti-LSV antibodies.

Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. CIM. Prod. Rev. 5:34-41 (1986)).

Further provided are methods for detecting a LSV nucleic acid molecule in a biological sample using an oligonucleotide probe and/or primer specific for the LSV nucleic acid molecules disclosed herein. In some embodiments, the method includes contacting the biological sample with an oligonucleotide probe that specifically hybridizes with a LSV nucleic acid molecule; and detecting hybridization of the probe with the biological sample. Hybridization of the probe to the biological sample, such as in high stringency or very high stringency conditions, indicates the presence of the LSV nucleic acid molecule in the biological sample.

In some embodiments, the biological sample is a nucleic acid amplification product obtained by a method comprising isolating RNA from the biological sample; reverse transcribing the RNA to generate cDNA; and amplifying the cDNA using a pair of primers that specifically hybridize to a LSV nucleic acid molecule, thereby producing a nucleic acid amplification product.

Also provided is a method for identifying a subject infected with a LSV using a pair of primers that specifically hybridize with a LSV nucleic acid molecule disclosed herein, such as an S, M or L LSV nucleic acid molecule. In some embodiments, the method includes isolating RNA from a biological sample obtained from the subject; reverse transcribing the RNA to generate cDNA; amplifying the cDNA using a pair of primers that specifically hybridize to a LSV nucleic acid molecule; and detecting an amplification product. Detection of the amplification product identifies the subject as infected with the LSV.

In some embodiments of the nucleic acid-based detection methods, the nucleotide sequence of the LSV nucleic acid molecule is at least 80%, at least 85%, at least 90%, at least 95%, at least 96%, at least 97%, at least 98% or at least 99% identical to SEQ ID NO: 1, 2, or 3, or a portion thereof is detected, such as, but not limited to, an open reading frame. In some examples, a nucleotide sequence of the Phlebovirus nucleic acid molecule comprising or consists of SEQ ID NO: 1, 2, or 3, or a portion thereof is detected. In some embodiments, a nucleic acid encoding one or more of SEQ ID NOs: 4-7 is detected.

In some embodiments of the nucleic acid-based detection methods, a pair of primers is used for nucleic acid amplification. In some embodiments, detecting the amplification product comprises hybridizing the amplification product to a probe. In some embodiments, the probe comprises a fluorophore, a quencher or both. In one non-limiting example, the probe comprises a fluorophore and two quenchers.

Methods of detecting specific nucleic acid molecules in a sample using polymerase chain reaction (PCR) are well known in the art. In some embodiments, the PCR detection method is a real-time PCR method, such as TAQMAN™ PCR. TAQMAN™ PCR assays typically use self-quenching probes, and in some cases, include a fluorophore and two quenchers. In some instances when a probe contains two quenchers, a first quencher is placed at the 3′ end of the probe and a second quencher is inserted into the oligonucleotide, such as by using a linker. The fluorophore is typically placed at the 5′ end of the oligonucleotide probe. During the annealing phase of each PCR cycle, the primers and double-quenched probe both bind complementary sections of the DNA. During the elongation phase, polymerization of the new DNA strand is initiated from the primers. Once the polymerase reaches the bound probe, its 5′ to 3′ exonuclease activity degrades the probe, thereby physically separating the quencher from the fluorophore. As a result, fluorescence can be measured and will increase in real-time with the exponential increase in PCR product.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided (e.g., in Innis et al. (1990) PCR Protocols, A Guide to Methods and Applications, Academic Press, Inc. N.Y.)).

The probes for LSV polynucleotides are of a length or have a sequence which allows the detection of unique viral sequences by hybridization. While about 6-8 nucleotides may be useful, longer sequences may be more effective, e.g., sequences of about 10-12 nucleotides, such as 11, 12, 13, 14, 15, 16, 17, 18, 19 or about 20 nucleotides or more. In some embodiments, these sequences will derive from regions which lack heterogeneity among viral isolates.

Non-PCR-based, sequence specific DNA amplification techniques can also be used with the invention to detect LSV sequences. An example of such techniques include, but are not limited to, the INVADER® assay (see, e.g., Kwiatkowski et al. Mol Diagn. December 1999, 4:353-64 and U.S. Pat. No. 5,846,717).

In other embodiments, solid substrates, such as arrays, are provided that include any of the polynucleotides described herein. The polynucleotides are immobilized on the arrays using methods known in the art. An array can have one or more different polynucleotides.

Probes (or sample nucleic acid) can be provided on an array for detection. Arrays can be created by, for example, spotting polynucleotide probes onto a substrate (e.g., glass, nitrocellulose, and the like) in a two-dimensional matrix or array. The probes can be bound to the substrate by either covalent bonds or by non-specific interactions, such as hydrophobic interactions. Samples of polynucleotides can be detectably labeled (e.g., using radioactive or fluorescent labels) and then hybridized to the probes. Double stranded polynucleotides, comprising the labeled sample polynucleotides bound to probe polynucleotides, can be detected once the unbound portion of the sample is washed away. Techniques for constructing arrays and methods of using these arrays are described in Published European Application No. EP 799 897; Published PCT Application No. WO 97/29212; Published PCT Application No. WO 97/27317; Published European Application No EP 785 280; Published PCT Application No. WO 97/02357; U.S. Pat. No. 5,593,839; U.S. Pat. No. 5,578,832; Published European Application No EP 728 520; U.S. Pat. No. 5,599,695; Published European Application No EP 721 016; U.S. Pat. No. 5,556,752; WO 95/22058; and U.S. Pat. No. 5,631,734. Arrays are particularly useful where, for example a single sample is to be analyzed for the presence of two or more nucleic acid target regions, as the probes for each of the target regions, as well as controls (both positive and negative) can be provided on a single array. Arrays thus facilitate rapid and convenience analysis.

### Immunogenic Compositions and Use Thereof

Any of the LSV polypeptides, polynucleotides, and recombinant viruses disclosed herein can be used in immunogenic compositions to elicit an immune response, such as to provide protection against infection by a LSV. Thus, the compositions disclosed herein can be used prophylactically or therapeutically. The compositions can be used to produce an immune response in a healthy subject or a subject infected with an LSV. The immunogenic composition optionally includes an adjuvant.

In some embodiments, immunogenic compositions are provide that include a LSV polypeptide as disclosed herein, or an immunogenic fragment thereof, and a pharmaceutically acceptable carrier. The immunogenic composition can further include an adjuvant. In some examples, the immunogenic composition comprises a LSV NSs, NP, GP or L protein. In particular examples, the amino acid sequence of the Phlebovirus polypeptide is at least 80%, at least 85%, at least 90%, at least 95%, at least 96%, at least 97%, at least 98% or at least 99% identical to an open reading from of SEQ ID NO: 1, the complement thereof, SEQ ID NO: 2 or SEQ ID NO: 3. In other particular examples, the amino acid sequence of the Phlebovirus polypeptide comprises or consists of an open reading from of SEQ ID NO: 1, the complement thereof, SEQ ID NO: 2 or SEQ ID NO: 3. In yet other examples, the polypeptide comprises an immunogenic fragment of an open reading from of SEQ ID NO: 1, the complement thereof, SEQ DI NO: 2, or SEQ ID NO: 3. In other examples, the immunogenic composition includes a polypeptide including, or consisting of, the amino acid sequence set forth as one of SEQ ID NOs: 4-7.

Also provided are immunogenic compositions comprising a recombinant LSV as described herein and a pharmaceutically acceptable carrier. The LSV can be attenuated.

In some embodiments, the recombinant LSV comprises an S segment having a nucleotide sequence at least 85%, at least 90%, at least 95%, at least 96%, at least 97%, at least 98% or at least 99% identical to SEQ ID NO: 1; an M segment having a nucleotide sequence at least 85%, at least 90%, at least 95%, at least 96%, at least 97%, at least 98% or at least 99% identical to SEQ ID NO: 2; and an L segment having a nucleotide sequence at least 85%, at least 90%, at least 95%, at least 96%, at least 97%, at least 98% or at least 99% identical to SEQ ID NO: 3.

Whole virus vaccines (live and attenuated, or replication incompetent, or killed) or subunit vaccines, such as structural or non-structural LSV proteins or immunogenic fragments thereof, can be used to treat or prevent LSV infections, respectively by eliciting an immune response in a subject. Alternatively, a pharmaceutical composition can comprise an antigen-presenting cell (e.g., a dendritic cell) transfected with a LSV polynucleotide such that the antigen-presenting cell expresses a LSV polypeptide, respectively.

Further provided is a method of eliciting an immune response against LSV in a subject by administering to the subject a therapeutically effective amount of a recombinant LSV, a LSV polypeptide, or nucleic acid molecule encoding an LSV polypeptide, or an immunogenic composition as disclosed herein. In some embodiments, the subject is administered the recombinant LSV, LSV polypeptide, or immunogenic composition prophylactically to prevent infection by a LSV. In other cases, the recombinant LSV, LSV polypeptide, nucleic acid molecule encoding an LSV polypeptide, or immunogenic composition is administered as a treatment for A LSV infection.

Also provided is a method of immunizing a subject against Phlebovirus infection, such as an LSV infection, by administering to the subject a therapeutically effective amount of a recombinant LSV, a LSV polypeptide, nucleic acid molecule encoding an LSV polypeptide, or an immunogenic composition as disclosed herein.

Nucleic acid vaccines encoding a genome, structural protein or non-structural protein or a fragment thereof of LSV can be used to elicit an immune response to treat or prevent LSV infection, respectively. Numerous gene delivery techniques are well known in the art, such as those described by Rolland (1998) Crit. Rev. Therap. Drug Carrier Systems 15:143-198, and references cited therein. Appropriate nucleic acid expression systems contain the necessary DNA sequences for expression in the patient (such as a suitable promoter and terminating signal). In a preferred embodiment, the DNA can be introduced using a viral expression system (e.g., vaccinia, pox virus, retrovirus, or adenovirus), which can involve the use of a non-pathogenic (defective), replication competent virus. Suitable systems are disclosed, for example, in Fisher-Hoch et al. (1989) Proc. Natl. Acad. Sci. USA 86:317-321; Flexner et al. (1989) Ann. N.Y. Acad. Sci. 569:86-103; Flexner et al. (1990) Vaccine 8:17-21; U.S. Pat. Nos. 4,603,112, 4,769,330, 4,777,127 and 5,017,487; PCT Publication No. WO 89/01973; Great Britain Publication No. 2,200,651; European Publication No. 0,345,242; PCT Publication No. WO 91/02805; Berkner (1988) Biotechniques 6:616-627; Rosenfeld et al. (1991) Science 252:431-434; Kolls et al. (1994) Proc. Natl. Acad. Sci. USA 91:215-219; Kass-Eisler et al. (1993) Proc. Natl. Acad. Sci. USA 90:11498-11502; Guzman et al. (1993) Circulation 88:2838-2848; and Guzman et al. (1993) Cir. Res. 73:1202-1207. Techniques for incorporating DNA into such expression systems are well known to those of ordinary skill in the art. The DNA can also be “naked,” as described, for example, in Ulmer et al. (1993) Science 259:1745-1749 and reviewed by Cohen (1993) Science 259:1691-1692. The uptake of naked DNA can be increased by coating the DNA onto biodegradable beads, which are efficiently transported into the cells. It will be apparent that a vaccine can comprise both a polynucleotide and a polypeptide component. Such vaccines can provide for an enhanced immune response.

Vaccine preparation is generally described in, for example, Powell and Newman, eds., Vaccine Design (the subunit and adjuvant approach), Plenum Press (NY, 1995). Vaccines can be designed to generate antibody immunity and/or cellular immunity such as that arising from CTL or CD4+ T cells.

A non-specific immune response enhancer can be any substance that enhances an immune response to an exogenous antigen. Examples of non-specific immune response enhancers include adjuvants, biodegradable microspheres (e.g., polylactic galactide) and liposomes (into which the compound is incorporated; see, e.g., U.S. Pat. No. 4,235,877). Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, can also be used as adjuvants. These are of use in inducing an immune response.

Pharmaceutical compositions and vaccines can also contain other compounds, which can be biologically active or inactive. For example, one or more immunogenic portions of other antigens can be present, either incorporated into a fusion polypeptide or as a separate compound, within the composition or vaccine. Polypeptides can, but need not be, conjugated to other macromolecules as described, for example, within U.S. Pat. Nos. 4,372,945 and 4,474,757. Pharmaceutical compositions and vaccines can generally be used for prophylactic and therapeutic purposes.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Aerosol formulations (i.e., they can be “nebulized”) are administered via inhalation. Aerosol formulations can be placed into pressurized acceptable propellants, such as dichlorodifluoromethane, propane, nitrogen, and the like.

Formulations suitable for parenteral administration, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Compositions can be administered, for example, by intravenous infusion, inhalation, parenterally, orally, topically, intradermally, intraperitoneally, intravenously, intravesically, rectally or intrathecally. The formulations can be presented in unit-dose or multi-dose sealed containers, such as ampules and vials. Pharmaceutically acceptable carriers are determined in part by the particular composition being administered (e.g., nucleic acid, protein, modulatory compounds or transduced cell), as well as by the particular method used to administer the composition. Accordingly, there are a wide variety of suitable formulations of pharmaceutical compositions of the present invention (see, e.g., Remington's Pharmaceutical Sciences, 17^{th} ed., 1989).

Such compositions can also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention can be formulated as a lyophilizate. Compounds can also be encapsulated within liposomes using well known technology.

Injection solutions and suspensions can be prepared from sterile powders, granules, and tablets of the kind previously described. Cells transduced by nucleic acids for ex vivo therapy can also be administered intravenously or parenterally.

The dose administered to a subject should be sufficient to affect a beneficial therapeutic response in the subject over time. The dose will be determined by the efficacy of the particular vector employed and the condition of the subject, as well as the body weight or surface area of the subject to be treated. The size of the dose also will be determined by the existence, nature, and extent of any adverse side-effects that accompany the administration of a particular vector, or transduced cell type in a particular patient.

For administration, compounds and transduced cells of the present invention can be administered at a rate determined by the LD-50 of the inhibitor, vector, or transduced cell type, and the side-effects of the inhibitor, vector or cell type at various concentrations, as applied to the mass and overall health of the patient. Administration can be accomplished via single or divided doses.

Pharmaceutical and vaccine compositions can be presented in unit-dose or multi-dose containers, such as sealed ampoules or vials. Such containers are preferably hermetically sealed to preserve sterility of the formulation until use. In general, formulations can be stored as suspensions, solutions or emulsions in oily or aqueous vehicles. Alternatively, a vaccine or pharmaceutical composition can be stored in a freeze-dried condition requiring only the addition of a sterile liquid carrier immediately prior to use.

### Assays for Modulators of LSV

Modulation of a LSV can be assessed using a variety of in vitro and in vivo assays, including cell-based models. Such assays can be used to test for inhibitors and activators of LSV. Modulators of LSV are tested using either recombinant or naturally occurring protein of choice. Modulation can include, but is not limited to, modulation of infection, replication, receptor binding, cell entry, particle formation, and the like.

Measurement of modulation of a LSV polypeptide, or a cell expressing LSV, either recombinant or naturally occurring, can be performed using a variety of assays, in vitro, in vivo, and ex vivo, as described herein. A suitable physical, chemical or phenotypic change that affects activity, e.g., enzymatic activity, cell surface marker expression, viral replication and proliferation can be used to assess the influence of a test compound on the polypeptide of this invention. When the functional effects are determined using intact cells or animals, one can also measure a variety of effects.

Assays to identify compounds with LSV modulating activity can be performed in vitro. Such assays can use full length LSV polypeptide or a variant thereof, or a mutant thereof, or a fragment thereof. Purified recombinant or naturally occurring protein can be used in the in vitro methods of the invention. The recombinant or naturally occurring protein can be part of a cellular lysate or a cell membrane. As disclosed below, the binding assay can be either solid state or soluble. Preferably, the protein or membrane is bound to a solid support, either covalently or non-covalently. Often, the in vitro assays of the invention are substrate or ligand binding or affinity assays, either non-competitive or competitive. Other in vitro assays include measuring changes in spectroscopic (e.g., fluorescence, absorbance, refractive index), hydrodynamic (e.g., shape), chromatographic, or solubility properties for the protein.

A high throughput binding assay can be performed in which the protein or a fragment thereof is contacted with a potential modulator and incubated for a suitable amount of time. In one embodiment, the potential modulator is bound to a solid support, and the protein is added. In another embodiment, the protein is bound to a solid support. A wide variety of modulators can be used, as described below, including small organic molecules, peptides, antibodies, etc. A wide variety of assays can be used to identify LSV-modulator binding, including labeled protein-protein binding assays, electrophoretic mobility shifts, immunoassays, enzymatic assays, and the like. In some cases, the binding of the candidate modulator is determined through the use of competitive binding assays, where interference with binding of a known ligand or substrate is measured in the presence of a potential modulator. Either the modulator, the known ligand, or substrate is bound first; then the competitor is added. After the protein is washed, interference with binding, either of the potential modulator or of the known ligand or substrate, is determined. Often, either the potential modulator or the known ligand or substrate is labeled.

A cell-based assay can be used in which the LSV is expressed in a cell, and functional, physical, chemical and phenotypic changes, such as vacuole formation, are assayed to identify viral modulators. Any suitable functional effect can be measured as described herein, in addition to viral inhibition assays as are well known in the art. The LSV be naturally occurring or recombinant. Also, fragments of the LSV or chimeric proteins can be used in cell based assays. In addition, point mutants in essential residues required by the catalytic site can be used in these assays.

In one embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., I Amer. Chem. Soc. 116:2661 (1994)), oligocarbamates (Cho et al., Science 261:1303 (1993)), and/or peptidyl phosphonates (Campbell et al., I Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996)), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

A solid state or soluble high throughput assaying using LSV, or a cell or tissue expressing a LSV protein can be used. A solid phase based in vitro assay can be used in a high throughput format can be used where LSV is attached to a solid phase. Any one of the assays described herein can be adapted for high throughput screening.

In high throughput assays, either soluble or solid state, it is possible to screen up to several thousand different modulators or ligands in a single day. This methodology can be used for investigating LSV in vitro, or for cell-based or membrane-based assays comprising a LSV. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-about 1500 different compounds. It is possible to assay many plates per day; assay screens for up to about 6,000, 20,000, 50,000, or more than 100,000 different compounds are possible using the integrated systems of the invention.

For a solid state reaction, the protein of interest or a fragment thereof, e.g., an extracellular domain, or a cell or membrane comprising the protein of interest or a fragment thereof as part of a fusion protein can be bound to the solid state component, directly or indirectly, via covalent or non-covalent linkage. A tag for covalent or non-covalent binding can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.). Antibodies to molecules with natural binders such as biotin are also widely available and appropriate tag binders (see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like (see, e.g., Pigott & Power, The Adhesion Molecule Facts Book I (1993)). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, polyethylene glycol linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature (e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates)). Non-chemical approaches for fixing tag binders to substrates include other common methods, such as heat, cross-linking by UV radiation, and the like.

The compounds tested as modulators of LSV can be any small organic molecule, or a biological entity, such as a protein, e.g., an antibody or peptide, a sugar, a nucleic acid, e.g., an antisense oligonucleotide or a ribozyme or siRNA, or a lipid. Alternatively, modulators can be genetically altered versions of a LSV. Typically, test compounds will be small organic molecules, peptides, circular peptides, siRNA, antisense molecules, ribozymes, and lipids.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

### Kits

Diagnostic reagents and kits are provided that include one or more such reagents for use in a variety of diagnostic assays, including for example, immunoassays such as ELISA and “sandwich” type immunoassays, as well as nucleic acid assay, e.g., PCR assays. In a related embodiment, the assay is performed in a flow-through or strip test format, wherein the binding agent is immobilized on a membrane, such as nitrocellulose.

The kit can include one or more probes or primers specific for a LSV nucleic acid sequence. The kit can include one or more antibodies, such as a monoclonal or polyclonal antibody that specifically binds a LSV polypeptide. The kit can also include one or more LSV polypeptides. The kit can include one or more LSV polynucleotides, such as cDNAs.

In several embodiments, such kits can include at least a first peptide, or a first antibody or antigen binding fragment of the invention, a functional fragment thereof, or a cocktail thereof, or a first nucleic acid molecule, and means for signal generation. The kit's components can be pre-attached to a solid support, or can be applied to the surface of a solid support when the kit is used. The signal generating means can come pre-associated with an antibody or nucleic acid of the invention or can require combination with one or more components, e.g., buffers, nucleic acids, antibody-enzyme conjugates, enzyme substrates, or the like, prior to use.

Kits can also include additional reagents, e.g., blocking reagents for reducing nonspecific binding to the solid phase surface, washing reagents, enzyme substrates, enzymes, and the like. The solid phase surface can be in the form of microtiter plates, microspheres, or other materials suitable for immobilizing nucleic acids, proteins, peptides, or polypeptides. An enzyme that catalyzes the formation of a chemiluminescent or chromogenic product or the reduction of a chemiluminescent or chromogenic substrate is one such component of the signal generating means. Such enzymes are well known in the art. Where a radiolabel, chromogenic, fluorigenic, or other type of detectable label or detecting means is included within the kit, the labeling agent can be provided either in the same container as the diagnostic or therapeutic composition itself, or can alternatively be placed in a second distinct container means into which this second composition can be placed and suitably aliquoted. Alternatively, the detection reagent and the label can be prepared in a single container means, and in most cases, the kit will also typically include a means for containing the vial(s) in close confinement for commercial sale and/or convenient packaging and delivery.

The kit can include one or more containers for storing a disclosed antibody, nucleic acid or polypeptide, as well as and a label or package insert on or associated with the container. Suitable containers include, for example, bottles, vials, syringes, etc. The containers may be formed from a variety of materials such as glass or plastic. The container holds a composition which is effective for diagnosis and/or treatment. In some embodiments, the container can have a sterile access port (for example the container may be an intravenous solution bag or a vial having a stopper pierceable by a hypodermic injection needle). The label or package insert indicates that the contents are used for treating the particular condition, or for detection/diagnosis.

In some embodiments, the kit includes instructional materials, such as the package insert, which discloses means of use of a LSV polypeptide, nucleic acid, or antibody. The instructional materials may be written, in an electronic form (such as a computer diskette or compact disk) or may be visual (such as video files). The instructions can include information on where to obtain detailed methods, such as disclosing internet link or reference to a website. The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method.

### Exemplary Embodiments

In some embodiments, an isolated nucleic acid molecule is disclosed that comprises, or consists of, a nucleic acid sequence at least 90% identical to an open reading frame of SEQ ID NO: 1, the complement thereof, SEQ ID NO: 2 or SEQ ID NO: 3. The isolated nucleic acid molecule can comprise, or consist of, an open reading frame of the nucleic acid sequence set forth as SEQ ID NO: 1, the complement thereof, SEQ ID NO: 2 or SEQ ID NO: 3. The isolated nucleic acid can comprise, or consist of, the nucleic acid sequence set forth as SEQ ID NO: 1, SEQ ID NO: 2 or SEQ ID NO: 3. The isolated nucleic acid molecule encodes a polypeptide comprising the nucleic acid sequence set forth as SEQ ID NOs: 4-7. The nucleic acid molecule can be a cDNA. These nucleic acids can be operably linked to a promoter and/or included in a vector. In additional embodiments, isolated polypeptides are disclosed that are encoded by any of these nucleic acid molecules. The nucleic acid molecules and vectors can be expressed in a host cell. A method of expressing a protein by culturing the host cell in vitro is also disclosed. An isolated polypeptide is also disclosed that can comprise, or consist of, an amino acid sequence at least 80% identical to an amino acid sequence set forth as one of SEQ ID NOs: 4-7, or can comprise or consist of the amino acid sequence set forth as one of SEQ ID NOs: 4-7.

Probes and primers are also disclosed. In some embodiments an isolated oligonucleotide is provided that is 12 to 40 nucleotides in length, wherein the oligonucleotide specifically hybridizes to one of SEQ ID NO: 1, SEQ ID NO: 2, or SEQ ID NO: 3, or their complement, under high stringency conditions. The oligonucleotide can include a fluorophore, a quencher or both.

In additional embodiments, an isolated monoclonal antibody or antigen-binding fragment thereof is provided. The monoclonal antibody or antigen-binding fragment that specifically binds to: (a) an amino acid sequence encoded by an open reading frame of a nucleic acid sequence at least 90% identical to the nucleic acid sequence set forth as one of SEQ ID NO: 1, the complement thereof, SEQ ID NO: 2 or SEQ ID NO: 3; (b) an amino acid sequence encoded by an open reading frame of a nucleic acid sequence at least 95% identical to the nucleic acid sequence set forth as one of SEQ ID NO: 1, the complement thereof, SEQ ID NO: 2 or SEQ ID NO: 3; or (c) an amino acid sequence encoded by an open reading frame of the nucleic acid sequence set forth as one of SEQ ID NO: 1, the complement thereof, SEQ ID NO: 2 or SEQ ID NO:3. Also provided is an antibody or antigen-binding fragment specifically binds to the amino acid sequence set forth as one of SEQ ID NOs: 4-7. In specific non-limiting examples, the antibody or antigen binding fragment can be labeled. In other non-limiting examples, the antibody or antigen binding fragment is chimeric.

In further embodiments, a method is disclosed for detecting a Lone Star Virus polypeptide in a biological sample. The method includes contacting the biological sample with an isolated monoclonal antibody or antigen-binding fragment thereof that specifically binds to: (i) an amino acid sequence encoded by an open reading frame of a nucleic acid sequence at least 90% identical to the nucleic acid sequence set forth as one of SEQ ID NO: 1, the complement thereof, SEQ ID NO: 2 or SEQ ID NO: 3; (ii) an amino acid sequence encoded by an open reading frame of a nucleic acid sequence at least 95% identical to the nucleic acid sequence set forth as one of SEQ ID NO: 1, the complement thereof, SEQ ID NO: 2 or SEQ ID NO: 3; or (iii) an amino acid sequence encoded by an open reading frame of the nucleic acid sequence set forth as one of SEQ ID NO: 1, the complement thereof, SEQ ID NO: 2 or SEQ ID NO: 3. The method also includes detecting binding of the antibody or antigen-binding fragment to the biological sample, wherein binding of the antibody or antigen-binding fragment to the biological sample indicates the presence of the Lone Star Virus or the Lone Star Virus polypeptide in the biological sample.

In some embodiments, a method is disclosed for detecting Lone Star Virus specific antibodies in a biological sample. The method includes (a) contacting the biological sample with a polypeptide comprising (i) an amino acid sequence encoded by an open reading frame of a nucleic acid sequence at least 90% identical to the nucleic acid sequence set forth as one of SEQ ID NO: 1, the complement thereof, SEQ ID NO: 2 or SEQ ID NO: 3; (ii) an amino acid sequence encoded by an open reading frame of a nucleic acid sequence at least 95% identical to the nucleic acid sequence set forth as one of SEQ ID NO: 1, the complement thereof, SEQ ID NO: 2 or SEQ ID NO: 3; or (iii) an amino acid sequence encoded by an open reading frame of the nucleic acid sequence set forth as one of SEQ ID NO: 1, the complement thereof, SEQ ID NO: 2 or SEQ ID NO: 3. The method also includes detecting binding of the polypeptide to the biological sample, wherein binding of the polypeptide to the biological sample indicates the presence of the Lone Star Virus-specific antibodies in a biological sample. In some non-limiting examples, the method includes contacting the biological sample with a polypeptide comprising the amino acid sequence set forth as one of SEQ ID NOs: 4-7.

In additional embodiments, methods are disclosed for detecting a Lone Star Virus nucleic acid molecule in a biological sample. The method includes (a) contacting the biological sample with an isolated oligonucleotide 12 to 40 nucleotides in length, wherein the oligonucleotide specifically hybridizes to one of SEQ ID NO: 1, SEQ ID NO: 2 or SEQ ID NO: 3 under high stringency conditions; and (b) detecting hybridization of the probe with the biological sample, wherein hybridization of the probe to the biological sample indicates the presence of the Lone Star Virus nucleic acid molecule in the biological sample. In some embodiments, the biological sample is a nucleic acid amplification product obtained by a method comprising: (i) isolating RNA from the biological sample; (ii) reverse transcribing the RNA to generate cDNA; and (iii) amplifying the cDNA using a pair of primers that specifically hybridize to the isolated nucleic acid molecule of claim 6, thereby producing a nucleic acid amplification product.

In further embodiments, a method is provided for identifying a subject infected with the Lone Star Virus, comprising: (a) isolating RNA from a biological sample obtained from the subject; (b) reverse transcribing the RNA to generate cDNA; (c) amplifying the cDNA using a pair of primers that specifically bind to a nucleic acid molecule disclosed herein; and (d) detecting an amplification product from step (c), wherein detection of the amplification product identifies the subject as infected with the Lone Star Virus. The amplification product can be detected by hybridizing the amplification product to a probe. The probe can include a fluorophore, a quencher or both.

In other embodiments, a kit is disclosed. The kit includes a container comprising at least one of: a) an isolated nucleic acid molecule; b) a primer that hybridizes to the nucleotide sequence set forth as SEQ ID NO:1, SEQ ID NO: 2, SEQ ID NO: 3 or their complement under highly stringent conditions; c) an isolated polypeptide; or d) an antibody. The kit includes instructions for use. The isolated nucleic acid molecule can include: i) a nucleic acid sequence at least 90% identical to an open reading frame of SEQ ID NO: 1, the complement thereof, SEQ ID NO: 2 or SEQ ID NO: 3; ii) an open reading frame of the nucleic acid sequence set forth as SEQ ID NO: 1, the complement thereof, SEQ ID NO: 2 or SEQ ID NO: 3; (iii) the nucleic acid sequence set forth as SEQ ID NO: 1, SEQ ID NO: 2 or SEQ ID NO: 3; or (iv) a cDNA encoding one of SEQ ID NOs: 4-7. The isolated polypeptide can include: (i) an amino acid sequence encoded by an open reading frame of a nucleic acid sequence at least 90% identical to the nucleic acid sequence set forth as one of SEQ ID NO: 1, the complement thereof, SEQ ID NO: 2 or SEQ ID NO: 3; (ii) an amino acid sequence encoded by an open reading frame of a nucleic acid sequence at least 95% identical to the nucleic acid sequence set forth as one of SEQ ID NO: 1, the complement thereof, SEQ ID NO: 2 or SEQ ID NO: 3; (iii) an amino acid sequence encoded by an open reading frame of the nucleic acid sequence set forth as one of SEQ ID NO: 1, the complement thereof, SEQ ID NO: 2 or SEQ ID NO: 3; or (iv) an immunogenic fragment thereof. The antibody can specifically bind to the polypeptide.

In yet other embodiments a method is disclosed for producing an antibody. The method includes: (a) immunizing a mammal with a polypeptide or a nucleic acid encoding the polypeptide, and isolating the antibody. The peptide includes, or consists of, (i) an amino acid sequence encoded by an open reading frame of a nucleic acid sequence at least 90% identical to the nucleic acid sequence set forth as one of SEQ ID NO: 1, the complement thereof, SEQ ID NO: 2 or SEQ ID NO: 3; (ii) an amino acid sequence encoded by an open reading frame of a nucleic acid sequence at least 95% identical to the nucleic acid sequence set forth as one of SEQ ID NO: 1, the complement thereof, SEQ ID NO: 2 or SEQ ID NO: 3; (iii) an amino acid sequence encoded by an open reading frame of the nucleic acid sequence set forth as one of SEQ ID NO: 1, the complement thereof, SEQ ID NO: 2 or SEQ ID NO: 3; or (iv) an immunogenic fragment of the polypeptide. The antibody that specifically binds: (i) the amino acid sequence encoded by an open reading frame of a nucleic acid sequence at least 90% identical to the nucleic acid sequence set forth as one of SEQ ID NO: 1, the complement thereof, SEQ ID NO: 2 or SEQ ID NO: 3; (ii) the amino acid sequence encoded by an open reading frame of a nucleic acid sequence at least 95% identical to the nucleic acid sequence set forth as one SEQ ID NO: 1, the complement thereof, SEQ ID NO: 2 or SEQ ID NO: 3; or (iii) the amino acid sequence encoded by an open reading frame of the nucleic acid sequence set forth as one of SEQ ID NO: 1, the complement thereof, SEQ ID NO: 2 or SEQ ID NO: 3. In some examples, the antibody specifically binds the amino acid sequence set forth as one of SEQ ID NOs: 4-7.

In more embodiments, an isolated Phlebovirus is disclosed that includes an S segment, an M segment and an L segment, wherein: (i) the nucleotide sequence of the S segment is at least 80% identical to SEQ ID NO: 1; (ii) the nucleotide sequence of the M segment is at least 80% identical to SEQ ID NO: 2; (iii) the nucleotide sequence of the L segment is at least 80% identical to SEQ ID NO: 3; or (iv) all (i), (ii) and (iii). In some examples, (i) the nucleotide sequence of the S segment is at least 90% identical to SEQ ID NO: 1; (ii) the nucleotide sequence of the M segment is at least 90% identical to SEQ ID NO: 2; (iii) the nucleotide sequence of the L segment is at least 90% identical to SEQ ID NO: 3; or (iv) all of (i), (ii) and (iii). In other examples, (i) the nucleotide sequence of the S segment is at least 95% identical to SEQ ID NO: 1 (ii) the nucleotide sequence of the M segment is at least 95% identical to SEQ ID NO: 2 (iii) the nucleotide sequence of the L segment is at least 95% identical to SEQ ID NO: 3; or (iv) all of (i), (ii) and (iii). In yet other examples, (i) the S segment comprises the nucleic acid sequence set forth as SEQ ID NO: 1; (ii) the M segment comprises the nucleic acid sequence set forth as SEQ ID NO: 2; (iii) the L segment comprises the nucleic acid sequence set forth as SEQ ID NO: 3; or (iv) all of (i), (ii) and (iii). In more examples, (i) the S segment consists of the nucleic acid sequence set forth as SEQ ID NO: 1; (ii) the M segment consists of the nucleic acid sequence set forth as SEQ ID NO: 2; (iii) the L segment consists of the nucleic acid sequence set forth as SEQ ID NO: 3; or (iv) all of (i), (ii) and (iii). The Phlebovirus can encode the polypeptides disclosed herein. The Phlebovirus can be attenuated.

In additional embodiments, an immunogenic composition is disclosed. The composition can include an effective amount of one or more of the Phlebovirus, the isolated polypeptide, the isolated nucleic acid, and the vector as described above. The immunogenic composition can include a pharmaceutically acceptable carrier. The immunogenic composition can be used to elicit an immune response against a Lone Star Virus in a subject. The subject can be infected with the Lone Star Virus. The subject can be healthy. In specific non-limiting examples, the subject is administered an attenuated Phlebovirus.

The following examples are provided to illustrate certain particular features and/or embodiments. These examples should not be construed to limit the disclosure to the particular features or embodiments described.

### EXAMPLES

The growth characteristics and genome of Lone Star virus (LSV), an unclassified bunyavirus originally isolated from the lone star tick Amblyomma americanum were characterized. Lone Star Virus (LSV) was able to infect both human (HeLa) and monkey (Vero) cells. Cytopathic effects were seen within 72 hours in both cell lines; vacuolization was observed in infected Vero, but not HeLa, cells. Viral culture supernatants were examined by unbiased deep sequencing and analysis using an in-house developed rapid computational pipeline for viral discovery, which definitively identified LSV as a phlebovirus. De novo assembly of the full genome revealed that LSV is highly divergent, sharing <61% overall amino acid identity with any other bunyavirus. Despite this sequence diversity, LSV was found by phylogenetic analysis to be part of a well-supported clade that includes members of the Bhanja group viruses, which are most closely related to SFSTV/HRTV. The genome sequencing of LSV is a critical first step in developing diagnostic tools to determine the risk of arbovirus transmission by A. americanum, a tick of growing importance given its expanding geographic range and competence as a disease vector. This study also underscores the power of deep sequencing analysis in rapidly identifying and sequencing the genomes of viruses of potential clinical and public health significance.

### Example 1

### Materials and Methods

An isolate of the Lone Star virus strain TMA 1381 was obtained (Centers for Disease Control and Prevention (CDC), Fort Collins, Colo.). Experiments using LSV were performed in Biosafety Level-2 (BSL-2) facilities.

An LSV suckling mouse brain passage 5 preparation was inoculated into T25 flasks of HeLa and Vero cells cultured in DMEM medium (Invitrogen, N.Y.) supplemented with 2% FBS (Atlas Biologicals, Fort Collins, Colo.). The multiplicity of infection was 1.3 pfu/cell. Cytopathic effect (CPE) was monitored at 24-hour intervals for 7 days. Viral titers were determined at 72 hours post-inoculation (hpi) using a plaque assay. Briefly, cell supernatant was diluted ten-fold and 100 μl was inoculated onto Vero cell monolayers in 6-well plates using a 0.5% agarose double overlay. The cells were visualized with neutral red added to the second overlay.

LSV-infected Vero cell cultures were extracted using two different protocols, one to purify RNA from viral particles (“virus purification protocol”) and the other to extract total RNA. First, 130 μL of viral culture supernatant was treated with Turbo DNase, Baseline Zero DNase, Benzonase, and RNase A (Roche, South San Francisco, Calif.), and then extracted with the QIAAMP ULTRASENS® Virus Kit (Qiagen, Valencia, Calif.). A second RNA extraction without pre-nuclease treatment was performed on 400 μL of supernatant using TRIZOL® reagent (Life Technologies, Foster City, Calif.). Each extraction was performed according to the manufacturer's protocol.

An LSV cDNA library for ILLUMINA® sequencing was prepared with the SCRIPTSEQ™ version 2 kit according to the manufacturer's protocol (Epicentre, Madison, Wis.). The input consisted of 30 ng of RNA from the first extraction and 80 ng from the second extraction. RNA was reverse-transcribed to cDNA, adaptors were ligated to the cDNA ends, and the cDNA was then amplified with 17 cycles of PCR. AMPURE™ XP beads (Beckman Coulter Genomics, Brea, Calif.) were used to remove primer and adaptor dimers as well as larger PCR fragments (>600 bp) from the amplified cDNA library. Library size distribution and concentration were determined using a High Sensitivity DNA kit on an Agilent Bioanalyzer 2100 instrument (Agilent, Santa Clara, Calif.) and a KAPA Library Quantification Kit (Kapa Biosystems, Woburn, Mass.), respectively. Approximately 10 pmol of library was used for 150-bp paired-end sequencing on an ILLUMINA® MISEQ® Sequencer (Illumina, Hayward, Calif.).

All computational analyses of deep sequencing data were performed on a 64-core 1U Quad AMD OPTERON™ 6200 computational server with 512 GB RAM and using the Ubuntu 12.04 LTS operating system. Raw deep sequencing reads were first “preprocessed” by trimming of primers, filtering to exclude low-quality and low-complexity sequences, and removing all residual sequences <50 bp in length (Delwart E L (2007) Rev Med Virol 17: 115-131; Kong Y (2011) Genomics 98: 152-153; Morgulis A, et al. (2006) J Comput Biol 13: 1028-1040). Preprocessed reads were then analyzed using a rapid computational pipeline incorporating the SNAP (Zaharia M, et al. (2011) arXiv 1111.5572v1) and RAPSearch (Ye Y, et al. (2011) BMC Bioinformatics 12: 159) aligners for alignment to nucleotide and protein databases, respectively (Naccache, et al., manuscript in preparation). The format of the pipeline was based on a computational subtraction approach used previously for detection of 2009 pandemic influenza A (Greninger et al. (2010) PLoS One 5: e13381) and a novel hemorrhagic fever virus from Africa (Grard et al. (2012) PLoS Pathog 8: e1002924). Briefly, nucleotide alignments to human (hg19) and bacterial databases in GENBANK® were performed at high-stringency cutoffs (edit distance d12 for SNAP) to exclude sequences corresponding to host and protein alignments to the viral GENBANK® database at low-stringency cutoffs (edit distance d28 for SNAP and 10⁻¹ for RAPSearch) to identify viral sequences. For these initial alignments, the advantage of SNAP and RAPSearch was a 10-1,000× increase in computational speed while maintaining comparable accuracy relative to existing algorithms such as BLASTn/BLASTx (Zaharia et al. (2011) arXiv 1111.5572v1; Ye et al. (2011) BMC Bioinformatics 12: 159; Altschul S F, et al. (1990) J Mol Biol 215: 403-410). Candidate viral sequences were subsequently confirmed as true by direct BLASTn alignment to the identified viral genus or species using an E-value cutoff of 1×10⁻⁸. The approximate computational times for the preprocessing, SNAP nucleotide alignment to human/bacterial/viral databases, and RAPSearch amino acid alignment to the viral protein database were 5 minutes, 10 minutes, and 135 minutes, respectively (2 h total time).

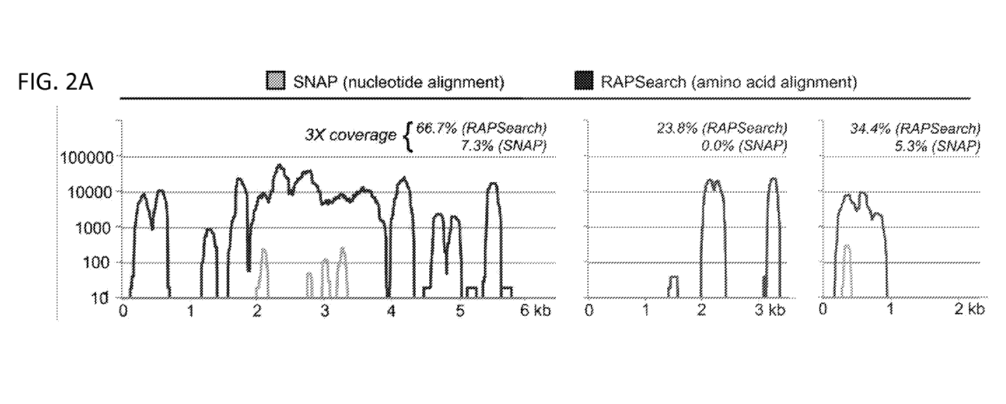

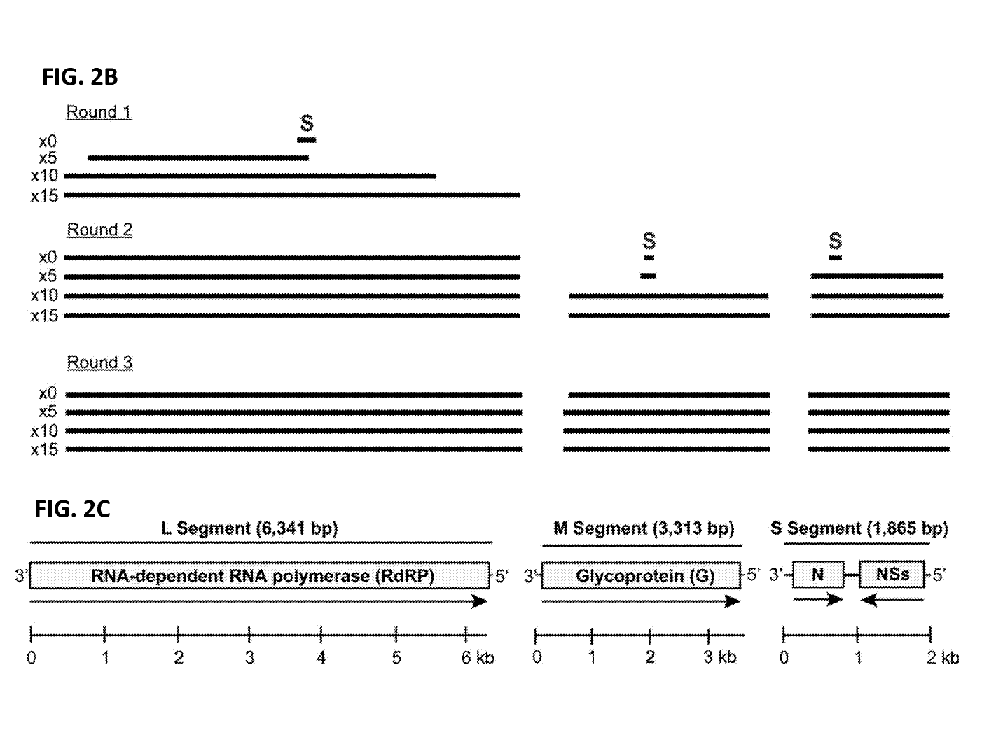

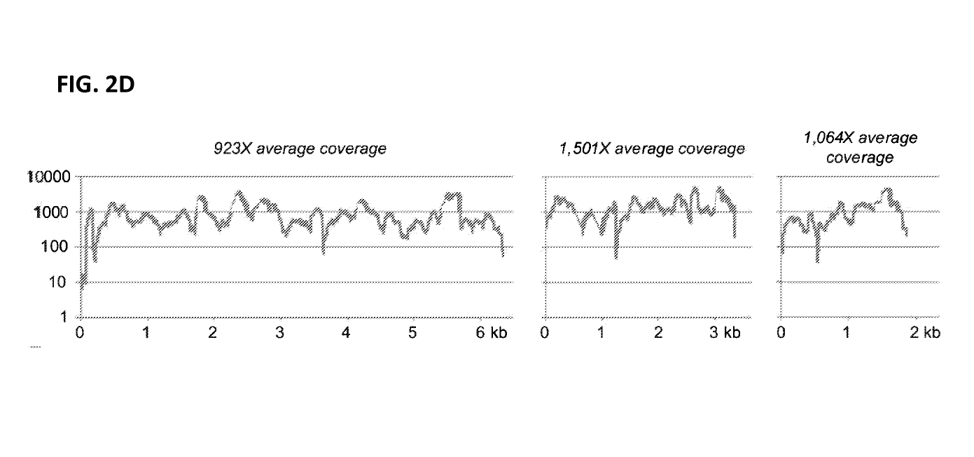

After running the deep sequencing reads through the computational pipeline, analysis of a subset of reads identified LSV as a highly divergent bunyavirus (Table 1). Coverage of the identified bunyavirus reads was not sufficient for genome assembly (4,188 of 6,341, 820 of 3,313, and 652 of 1,867, or 66.0%, 24.8%, and 34.9% of the L, M, and S segments at 3× coverage depth) (FIG. 2). Thus, a sequence “seed” was chosen from each of the presumptive L, M, and S segments and iterative de novo contig assembly using the entire preprocessed dataset was performed using the PRICE assembler (approximately 6 h total time) (Earl et al. (2011) Genome Res 21: 2224-2241; Ruby J G, DeRisi J L (2013) available at the derisilab.ucsf.edu website, sccessed January, 2012). Computational finishing of large contigs was then done manually using Geneious software v6.0 (Kearse et al. (2012) Bioinformatics 28: 1647-1649). The sequence of the 3′ end of the L segment of LSV, assembled with only 7× coverage (FIG. 2D), was confirmed using RACE (rapid amplification of cDNA ends)-PCR (Elbeaino et al. (2009) J Gen Virol 90: 1281-1288).

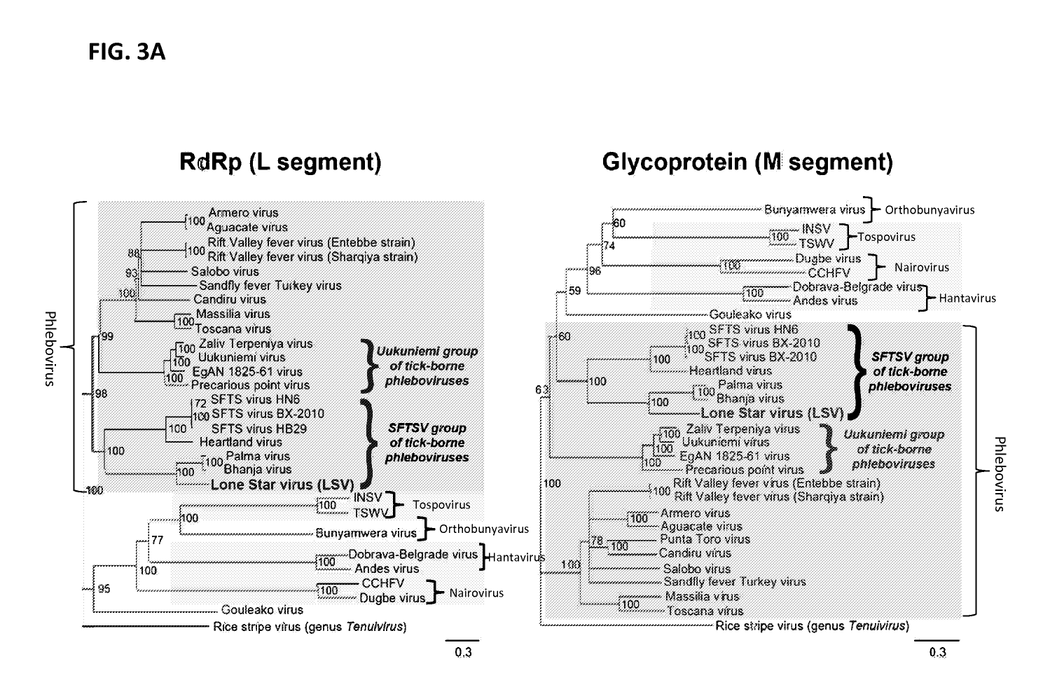

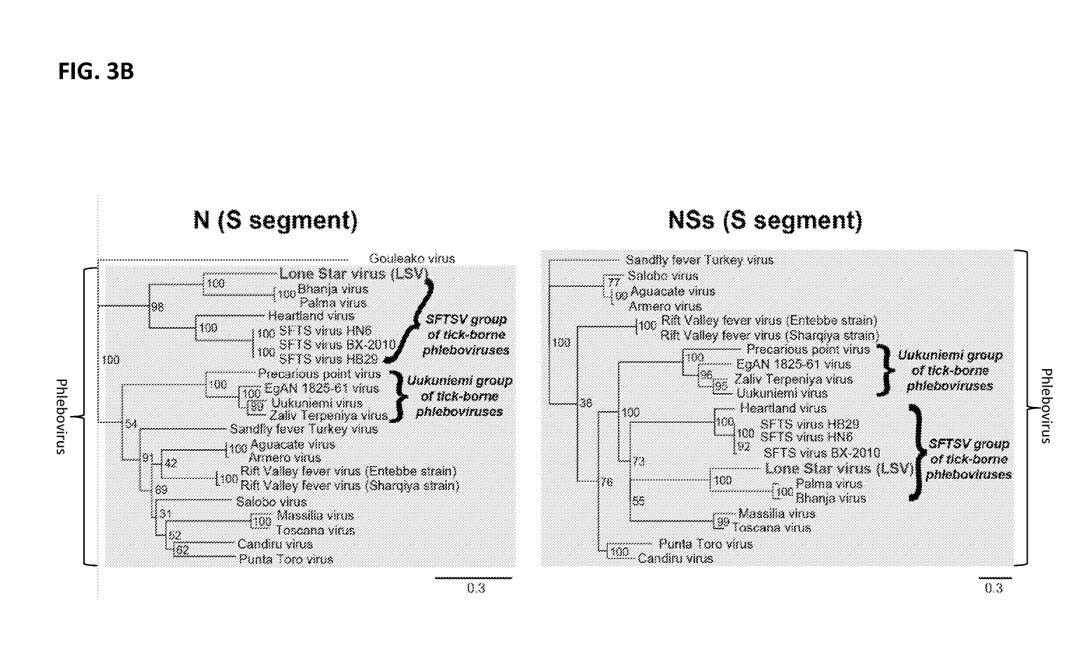

Phylogenetic analysis was performed on the 4 bunyavirus proteins: RdRp (L segment), glycoprotein (M segment), N protein (S segment), and NSs protein (S segment). Multiple sequence alignments of each of the LSV proteins relative to the corresponding protein from nearly all available phlebovirus sequences in GENBANK® and from Gouleako virus (Marklewitz et al. (2011) J Virol 85: 9227-9234) were first generated using MAFFT (v6.0) with the E-INS-I algorithm and at default settings (Katoh et al. (2005) Nucleic Acids Res 33: 511-518). Gouleako virus, a member of a novel bunyavirus genus and the closest known relative to phleboviruses (Marklewitz et al. (2011) J Virol 85: 9227-9234), was chosen as the outgroup for the RdRp, glycoprotein, and N protein trees. Phylogenetic trees and bootstrap confidence levels after 10,000 bootstrapping replicates were determined in Geneious using a Jukes-Cantor model and the neighbor-joining method at a support threshold of 25% (FIG. 3). Tree topologies were then confirmed using an alternate maximum likelihood Bayesian approach with MrBayes V3.2 software (20,000 sample trees, 25% of trees discarded as burn-in) (Ronquist et al. (2012) Syst Biol 61: 539-542). The overall tree topologies using neighbor joining or maximum likelihood approaches were the same.

Deduced amino acid sequences of LSV were compared by pairwise sliding window alignments in Geneious (Kearse et al. (2012) Bioinformatics 28: 1647-1649). Specifically, LSV was aligned to Bhanja virus strain IG690, Heartland virus, the three SFTS phlebovirus isolates from China (SFTS BX-2010, SFTS virus HB29, and SFTS virus HN6), Uukuniemi virus, Rift Valley Fever virus strain Sharqiya, and CCHF virus, using MAFFT (v6.0) with the FFT-NS-I x1000 algorithm at default settings (Katoh et al. (2005) Nucleic Acids Res 33: 511-518), and pairwise identities were plotted across each viral segment using a sliding window of 50 amino acids. Overall amino acid pairwise similarity was determined by concatenating the 4 bunyavirus protein sequences and running pairwise alignments in Geneious.

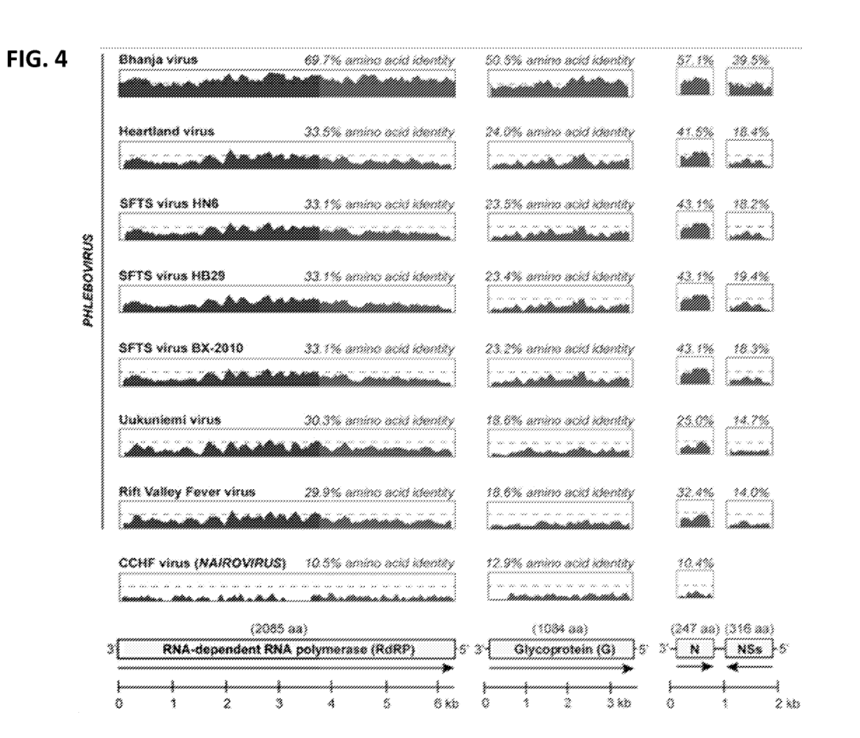

GENBANK® accession numbers used for FIGS. 3 and 4 are as follows: RdRp (L segment): Aguacate virus (NC_015451), Armero virus (HQ661805), Bhanja virus strain ibAr2709 (JX961616), Bhanja virus strain IG690 (JX961619), Bhanja virus strain M3811 (JQ956376), Bhanja virus strain R-1819 (JX961622), Candiru virus (NC_015374), CCHF virus (NC_005301), EgAN 1825-61 virus (HM566159), Gouleako virus (HQ541738), Heartland virus (JX005847), Massilia virus (EU725771), Palma virus strain PoTi4.92 (JX961628), Palma virus strain M3443 (JQ956379), Precarious point virus (HM566181), Rift Valley fever strain Sharqiya (NC_014397), Rift Valley fever virus strain Smithburn (DQ375430), Salobo virus (HM627185), Tosacana virus (NC_006319), Sandfly fever Turkey virus (NC_015412), SFTS virus BX-2010 (JF682773), SFTS virus HB29 (NC_018136), SFTS virus HN6 (HQ141595), Uukuniemi virus (UUKLRNAP), Zaliv Terpeniya virus (HMX66191); glycoprotein (M segment): Aguacate virus (NC_015450), Armero virus (HQ661806), Bhanja virus strain ibAr2709 (JX961616), Bhanja virus strain IG690 (JX961620), Bhanja virus strain M3811 (JQ956377), Bhanja virus strain R-1819 (JX961620), Candiru virus (NC_015373), EgAN 1825-61 virus (HM566158), CCHF virus (NC_005300), Gouleako virus (HQ541737), Heartland virus (JX005845), Massilia virus (EU725772), Palma virus strain PoTi4.92 (JX961629), Palma virus strain M3443 (JQ956380), Precarious point virus (HM566179), Rift Valley fever strain Sharqiya (NC_014396), Rift Valley fever virus strain Smithburn (DQ80193), Salobo virus (HM627183), Tosacana virus (NC_006320), Sandfly fever Turkey virus (NC_015411), SFTS virus BX-2010 (JF682774), SFTS virus HB29 (NC_018138), SFTS virus HN6 (HQ141596), Uukuniemi virus (UUKGPM), Zaliv Terpeniya virus (HMX66193); N (S segment): Aguacate virus (NC_015452), Armero virus (HQ661807), Bhanja virus strain ibAr2709 (JX961618), Bhanja virus strain IG690 (JX961621), Bhanja virus strain M3811 (JQ956378), Bhanja virus strain R-1819 (JX961624), Candiru virus (NC_015375), CCHF virus (NC_005302), EgAN 1825-61 virus (HM566160), Gouleako virus (HQ541736), Heartland virus (JX005843), Massilia virus (EU725773), Palma virus strain PoTi4.92 (JX961630), Palma virus strain M3443 (JQ956381), Precarious point virus (HM566180), Rift Valley fever strain Sharqiya (NC_014396), Rift Valley fever virus strain Smithburn (DQ80157), Salobo virus (HM627184), Tosacana virus (NC_006318), Sandfly fever Turkey virus (NC_015413), SFTS virus BX-2010 (JF682775), SFTS virus HB29 (NC_018137), SFTS virus HN6 (HQ141597), Uukuniemi virus (UUKNNSA), Zaliv Terpeniya virus (HMX66192); NSs (S segment): Aguacate virus (NC_015452), Armero virus (HQ661807), Bhanja virus strain ibAr2709 (JX961618), Bhanja virus strain IG690 (JX961621), Bhanja virus strain M3811 (JQ956378), Bhanja virus strain R-1819 (JX961624), Candiru virus (NC_015375), EgAN 1825-61 virus (HM566160), Heartland virus (JX005843), Massilia virus (EU725773), Palma virus strain PoTi4.92 (JX961630), Palma virus strain M3443 (JQ956381), Precarious point virus (HM566180), Rift Valley fever strain Sharqiya (NC_014396), Rift Valley fever virus strain Smithburn (DQ80157), Salobo virus (HM627184), Tosacana virus (NC_006318), Sandfly fever Turkey virus (NC_015413), SFTS virus BX-2010 (JF682775), SFTS virus HB29 (NC_018137), SFTS virus HN6 (HQ141597), Uukuniemi virus (UUKNNSA), Zaliv Terpeniya virus (HMX66192). All GENBANK® accession numbers listed above are incorporated herein by reference as available on Apr. 19, 2013.

The nucleic acid and amino acid sequences are disclosed in GENBANK® Accession No. NC_021242.1, May 21, 2013; GENBANK® Accession No. KC589005.1, GENBANK® Accession No. May 18, 2013; GENBANK® Accession No. NC_021244.1, May 21, 2013, GENBANK® Accession No. NC_021243.1, May 21, 2013; GENBANK® Accession No. KC589007.1, May 18, 2013; and GENBANK® Accession No. KC589006.1, May 18, 2013, which are all incorporated by reference herein in their entirety.

### Example 2

### Cellular Effects

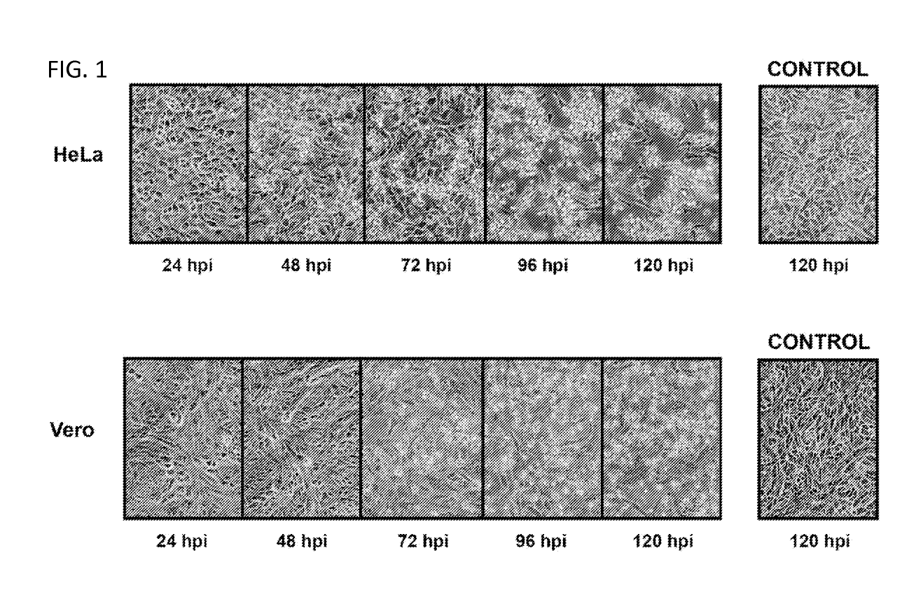

Lone Star virus (LSV) induced cytopathic effect (CPE) in both non-human primate (Vero) and human (HeLa) cell types at 72 hours post-inoculation (hpi) (FIG. 1). Nearly complete clearing of the cell sheet was observed with infected HeLa cells, but not with Vero cells, at 96 hpi. Inoculated Vero cells developed vacuoles starting at 72 hpi with increasing abundance as CPE progressed. Vacuoles were not observed with inoculated HeLa cells. The titers of infectious LSV produced in HeLa and Vero cell culture at 72 hpi in plaque-forming units (PFU) per milliliter were 1.9×10⁶ PFU/ml and 1.2×10⁶ PFU/ml, respectively.

### Example 3

### Sequencing

Unbiased next-generation or “deep sequencing” was then used to analyze LSV culture supernatants and assemble the viral genome. Since standard virus purification protocol using nuclease prior to extraction resulted in a very low concentration of RNA (less than 5 ng/μL), this material was pooled with RNA from a second extraction without nuclease treatment for cDNA deep sequencing library preparation. The final count of raw deep sequencing reads was 15,134,328 total sequences (7,567,164 150-bp paired-end sequences). Using a rapid computational pipeline developed in-house for pathogen identification from deep sequencing data, the dataset was comprehensively analyzed for viruses within 2 hours using a single 64-core computational server with 512 GB RAM. First, NGS reads were sequentially preprocessed, aligned to a reference human database, and aligned to a reference bacterial database, with removal of 50.3% (n=7,606,235), 15.5% (n=2,345,988), and 1.9% (n=293,410), respectively, of the raw sequences in the dataset. The remaining 4,888,695 sequences, comprising 32.3% of the original dataset, were then aligned to reference viral nucleotide and protein databases to identify reads corresponding to viruses (Table 1). Among the detected viruses, the vast majority were known contaminants in cell cultures, reagents, and the laboratory environment (Table 1).

TABLE 1
Deep sequencing reads matching to viral sequences

| | | | | # (%) of viral | |
|---|---|---|---|---|---|
| | | # of viral | # of viral | reads by | |
| | | reads by | reads by | RAPSearch + | |
| Virus | Viral Family | SNAP* | RAPSearch* | SNAP* | Presumed source |
| Simian | Retroviridae | 1,217 | 1,502 | 2,008 (5%) | Vero cell |
| endogenous | | | | | endogenous |
| retrovirus | | | | | retrovirus |
| Avian | Retroviridae | 608 | 833 | 936 (2.3%) | Known viral |
| myeloblastosis | | | | | contaminant of |
| virus | | | | | reverse |
| | | | | | transcriptase in |
| | | | | | ScriptSeq kit |
| Phlebovirus | Bunyaviridae | 90 | 37,292 | 37,322 (92.2%) | Culture of LSV |
| (LSV) | | | | | |
| Bovine viral | Flaviviridae | 93 | 139 | 148 (0.37%) | Contaminant of |
| diarrhea virus | | | | | fetal bovine serum |
| | | | | | used in virus |
| | | | | | culturing |
| Environmental | various | 4 | 73 | 75 (0.19%) | Environmental |
| DNA viruses | families* | | | | and/or reagent |
| | | | | | contamination |
| TOTAL (%) | | 2,012 | 39,839 | 40,489 (100% of | |
| | | | | viral reads, | |
| | | | | 0.27% of total | |
| | | | | reads) | |

*Viral reads were identified by comparison to viral nucleotide and amino acid databases using the SNAP (Zaharia M, et al. (2011) arXiv 1111.5572v1) and RAPSearch (Ye et al. (2011) BMC Bioinformatics 12: 159) aligners, respectively. Viral hits were confirmed to be true by BLASTn alignment to LSV or the closest viral genus or species in GENBANK ® using an E-value cutoff of 1 × 10⁻⁸. Out of 15,134,328 total reads, 40,489 reads (0.27%) were identified as viral by SNAP and/or RAPSearch. Out of these 40,489 viral hits, 37,322 (92.2%) corresponded to LSV. The actual number of LSV reads in the dataset is 142,941 (0.94% of the total reads); thus, only 26.1% (37,322 of 142,941) of the actual number of LSV reads was detected.

The sole exception was a phlebovirus in the Bunyaviridae family, reads from which comprised 92.2% of the viral deep sequencing hits. The phlebovirus reads represented all 3 segments and were highly divergent, sharing <50% identity with any other bunyavirus sequence in GENBANK®. As the alignable reads represented less than 50% overall coverage of the genome (FIG. 2A), the full LSV genome was subsequently recovered by 3 rounds of 15-cycle de novo assembly using a single “seed” corresponding to an identified read for each of the presumptive L, M, and S segments (FIG. 2B). Subsequent mapping of the preprocessed deep sequencing reads to the full LSV genome at high stringency showed that the actual coverage achieved averaged 1,112× [range 7-4,939×] (FIG. 2C). The mapping also revealed that the computational pipeline had detected only 26.1% (37,322/142,941) of the total number of LSV reads actually present in the deep sequencing data (Table 1).

As in other bunyaviruses, the genome of LSV consists of 3 negative-sense RNA segments (L (SEQ ID NO: 3), M (SEQ ID NO: 2), and S (SEQ ID NO: 1)) (FIG. 2B). These segments were found to contain coding sequences for the RdRp, G, N, and NSs proteins. The sizes of the L, M, and S segments of LSV are 6,341, 3,313, and 1,876 nt, respectively, encoding 2,085 amino acid (aa) L, 1,084 aa G, 247 aa N, and 316 aa NSs proteins. By phylogenetic analysis, LSV was found to be a member of the Phlebovirus genus, and part of a well-supported clade containing the recently sequenced Bhanja and Palma viruses (FIG. 3, “Bhanja”) (Dilcher et al. (2012) Virus Genes 45: 311-315; Matsuno et al. (2013) J Virol.). This clade was distinct from the SFTS group of tick-borne phleboviruses, which includes SFTSV and HRTV (FIG. 3, “SFTS”) (Xu B, et al. (2011) PLoS Pathog 7: e1002369; Yu X J, et al. (2011) N Engl J Med 364: 1523-1532; Zhang Y Z, et al. (2012) Clin Infect Dis 54: 527-533.), and the Uukunemi group, which includes among its members the Zaliv Terpeniya, Uukuniemi, EgAN 1825-61, and Precarious point viruses (FIG. 3, “Uukuniemi”) (Palacios et al. (2013) J Virol.), although it is more closely related phylogenetically to the SFTS than Uukunemi group.

The termini of the L, M, and S segments of LSV retain the conserved “5-ACACAAAG” and “CUUUGUGU-3” inverted signature sequences common to phleboviruses in the Bunyaviridae family, with the notable exception of the 5′ end of the S segment, which contains a “5′-ACACAGAG” sequence. This deviation from absolute conservation of the terminal signature sequences is also seen in other tick-borne phleboviruses, including SFTS, Heartland, Bhanja, and Palma viruses (Dilcher et al. (2012) Virus Genes 45: 311-315). Pairwise identity plots revealed that LSV is highly divergent with ≦61% overall amino acid identity to other representative bunyaviruses (FIG. 4). The closest relatives to LSV were the Bhanja and Palma viruses, with 39-70% amino acid identity across the 4 bunyavirus proteins (FIG. 4), with the next nearest neighbors, the SFTSV and Heartland viruses, sharing only 18-43% amino acid identity.

Phleboviruses that are pathogenic to humans can be transmitted by mosquitoes, sand flies, and ticks (Yu et al. (2011) N Engl J Med 364: 1523-1532). The A. americanum tick is well-suited as a zoonotic disease vector because it feeds on a wide assortment of wild animal hosts as well as humans. It is a vector for the pathogenic agents of Rocky Mountain spotted fever (Rickettsia rickettsia), human monocytic ehrlichiosis (Ehrlichia chaffeensis), and tularemia (Francisella tularensis), and has also been associated with Southern Tick-borne Rash Illness (STARI), for which no etiologic agent has been identified (Goddard and Varela-Stokes (2009) Vet Parasitol 160: 1-12). Because the geographic range of A. americanum is expanding northward in the eastern United States and it has a propensity for biting humans (Paddock and Yabsley (2007) Curr Top Microbiol Immunol 315: 289-324), its potential role as a vector of disease is increasingly important. However, the ability of A. americanum to harbor viruses is not well understood. The sequencing of LSV is thus a critical first step in determining the risk of arbovirus transmission by A. americanum.

The result presented herein show that LSV is a member of the Bhanja clade of tick-borne phleboviruses (FIG. 3) (Matsuno et al. (2013) J Virol.), which shares greater phylogenetic similarity to members of the SFTS than Uukuniemi clade (FIGS. 3 and 4). Despite high sequence divergence, serological cross-reactivity was recently observed between members of all three clades (Matsuno et al. (2013) J Virol.; Palacios G, et al. (2013) J Virol.). This suggests that members of the SFTS, Bhanja, and Uukuniemi clades comprise part of a single larger serogroup of tick-borne phleboviruses. As phleboviruses in the SFTS and Bhanja clades are known to be pathogenic in humans (Xu et al. (2011) PLoS Pathog 7: e1002369; Yu et al. (2011) N Engl J Med 364: 1523-1532; Zhang et al. (2012) Clin Infect Dis 54: 527-533; Calisher and Goodpasture (1975) Am J Trop Med Hyg 24: 1040-1042; Punda V, et al. (1980) Zentralblatt fur Bakteriologie: 297-301; Vesenjak-Hirjan J, et al. (1980) Zentralblatt fur Bakteriologie: 297-301), LSV can be associated with tick-borne illness in humans.

Both human (HeLa) and monkey (Vero) cells were found to support infection by LSV, suggesting that LSV is capable of infecting human and other nonhuman primates in vivo. However, although CPE was observed in both HeLa and Vero cells starting at 72 hpi, the appearance and progression of the CPE was different between the two LSV-inoculated cell lines. In particular, vacuoles were observed only in Vero cells. Similarly, cellular vacuoles presumably containing infectious viral particles were also seen with SFSTV and Heartland virus, which also grow efficiently in Vero cells (Yu et al. (2011) N Engl J Med 364: 1523-1532; McMullan et al. (2012) N Engl J Med 367: 834-841).

Identifying pathogens and then rapidly assembling their genomes de novo is challenging for widely divergent sequences such as those found in novel emerging viruses. When related genomes in large sequence databases such as GENBANK® are lacking, routine algorithms to map/align reads to reference sequences are inadequate. To address these challenges, a computational pipeline was developed for rapid identification and de novo genome assembly of viral pathogens from deep sequencing metagenomic datasets. After preprocessing and removal of sequences corresponding to host background and/or laboratory contamination, this pipeline incorporates both nucleotide and amino acid alignments to reference databases using efficient and highly parallelizable algorithms to comprehensively identify both known and novel viruses within hours. Protein alignments are critical for detecting highly divergent viruses such as LSV, as shown by the detection of ˜414× the number of phlebovirus reads by amino acid rather than nucleotide alignments (37,292 vs. 90, Table 1). In fact, even with the use of low-stringency amino acid alignments, the computational pipeline was only able to identify 26.1% of the total number of LSV reads actually present in the deep sequencing dataset (Table 1) due to the high sequence divergence of LSV relative to other bunyaviruses (FIG. 4). Thus, for genomic assembly, downstream “seed-based” de novo assembly packages such as PRICE® are useful for recovering full viral genomes in the absence of a closely related reference sequence (FIG. 2A) (Earl and Bradnam et al. (2011) Genome Res 21: 2224-2241.; Ruby and DeRisi (2013), available from on the internet from the derisilab.ucsf.edu website, ccessed January, 2012).

The incidence of new infectious diseases continues to increase. Vector-borne diseases accounted for nearly 30% of the emerging infectious disease events in the past decade (Jones et al. (2008) Nature 451: 990-993). The ability to rapidly identify emerging vector-borne pathogens for surveillance or outbreak investigation is an important part of understanding and dealing with these new diseases. A deep sequencing-based approach was developed for the rapid identification and de novo sequence assembly of a highly divergent phlebovirus in the A. americanum tick.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. An isolated nucleic acid molecule comprising a nucleic acid sequence at least 90% identical to an open reading frame of SEQ ID NO: 1, the complement thereof, SEQ ID NO: 2 or SEQ ID NO: 3.

2. The isolated nucleic acid molecule of claim 1, comprising an open reading frame of the nucleic acid sequence set forth as SEQ ID NO: 1, the complement thereof, SEQ ID NO: 2 or SEQ ID NO: 3.

3. The isolated nucleic acid molecule of claim 6, comprising the nucleic acid sequence set forth as SEQ ID NO: 1, SEQ ID NO: 2 or SEQ ID NO: 3.

4. The isolated nucleic acid molecule of claim 1, wherein the nucleic acid encodes a polypeptide comprising the amino acid sequence set forth as SEQ ID NOs: 4-7.

5. A cDNA comprising:
(a) a nucleotide sequence at least 90% identical to an open reading frame of SEQ ID NO: 1, the complement thereof, SEQ ID NO: 2 or SEQ ID NO: 3;
(b) an open reading frame of the nucleic acid sequence set forth as SEQ ID NO: 1, the complement thereof, SEQ ID NO: 2 or SEQ ID NO: 3; or
(c) a nucleic acid sequence encoding a polypeptide comprising the amino acid sequence set forth as SEQ ID NOs: 4-7.

6. An isolated polypeptide encoded by the nucleic acid molecule of claim 1.

7. The isolated polypeptide of claim 6, comprising an amino acid sequence at least 80% identical to an amino acid sequence set forth as one of SEQ ID NOs: 4-7.

8. The isolated polypeptide of claim 7, comprising the amino acid sequence set forth as one of SEQ ID NOs: 4-7.

9. The isolated nucleic acid molecule of claim 1, operably linked to a heterologous promoter.

10. A recombinant vector comprising the nucleic acid molecule of claim 9.

11. An isolated host cell comprising the vector of claim 10.

12. (canceled)

13. An isolated oligonucleotide 12 to 40 nucleotides in length, wherein the oligonucleotide specifically hybridizes to one of SEQ ID NO: 1, SEQ ID NO: 2, or SEQ ID NO: 3, or their complement, under high stringency conditions, and wherein the oligonucleotide comprises a fluorophore, a quencher or both.

14. An isolated monoclonal antibody or antigen-binding fragment thereof that specifically binds to:
(a) an amino acid sequence encoded by an open reading frame of a nucleic acid sequence at least 90% identical to the nucleic acid sequence set forth as one of SEQ ID NO: 1, the complement thereof, SEQ ID NO: 2 or SEQ ID NO: 3;
(b) an amino acid sequence encoded by an open reading frame of a nucleic acid sequence at least 95% identical to the nucleic acid sequence set forth as one of SEQ ID NO: 1, the complement thereof, SEQ ID NO: 2 or SEQ ID NO: 3; or
(c) an amino acid sequence encoded by an open reading frame of the nucleic acid sequence set forth as one of SEQ ID NO: 1, the complement thereof, SEQ ID NO: 2 or SEQ ID NO:3.

15. The antibody or antigen-binding fragment of claim 14, wherein the antibody or antigen-binding fragment specifically binds to the amino acid sequence set forth as one of SEQ ID NOs: 4-7.

16. The antibody of antigen binding fragment of claim 14, wherein the antibody or antigen binding fragment is labeled.

17. The antibody or antigen binding fragment of claim 14, wherein the antibody or antigen binding fragment is chimeric.

18. A method for detecting a Lone Star Virus polypeptide in a biological sample, comprising:
(a) contacting the biological sample with the isolated monoclonal antibody or antigen-binding fragment of claim 14; and
(b) detecting binding of the antibody or antigen-binding fragment to the biological sample, wherein binding of the antibody or antigen-binding fragment to the biological sample indicates the presence of the Lone Star Virus or the Lone Star Virus polypeptide in the biological sample.

19. A method for detecting Lone Star Virus specific antibodies in a biological sample, comprising:
(a) contacting the biological sample with the isolated polypeptide of claim 7;
(b) detecting binding of the polypeptide to the biological sample, wherein binding of the polypeptide to the biological sample indicates the presence of the Lone Star Virus-specific antibodies in a biological sample.

20. The method of claim 19, comprising contacting the biological sample with a polypeptide comprising the amino acid sequence set forth as one of SEQ ID NOs: 4-7.

21-22. (canceled)

23. A method for identifying a subject infected with the Lone Star Virus, comprising:
(a) isolating RNA from a biological sample obtained from the subject;
(b) reverse transcribing the RNA to generate cDNA;
(c) amplifying the cDNA using a pair of primers that specifically hybridize to the isolated nucleic acid molecule of claim 1 of the complement thereof; and
(d) detecting an amplification product from step (iii), wherein detection of the amplification product identifies the subject as infected with the Lone Star Virus.

24. The method of claim 23, wherein detecting the amplification product comprises hybridizing the amplification product to a probe.

25. The method of claim 24, wherein the probe comprises a fluorophore, a quencher or both.

26. A kit, comprising
a container comprising at least one of:
a) an isolated nucleic acid molecule comprising
i) a nucleic acid sequence at least 90% identical to an open reading frame of SEQ ID NO: 1, the complement thereof, SEQ ID NO: 2 or SEQ ID NO: 3;
ii) an open reading frame of the nucleic acid sequence set forth as SEQ ID NO: 1, the complement thereof, SEQ ID NO: 2 or SEQ ID NO: 3;
(iii) the nucleic acid sequence set forth as SEQ ID NO: 1, SEQ ID NO: 2 or SEQ ID NO: 3; or
(iv) a cDNA encoding one of SEQ ID NOs: 4-7;
b) a primer that hybridizes to the nucleotide sequence set forth as SEQ ID NO:1, SEQ ID NO: 2, SEQ ID NO: 3 or their complement under highly stringent conditions,
c) an isolated polypeptide comprising:
(i) an amino acid sequence encoded by an open reading frame of a nucleic acid sequence at least 90% identical to the nucleic acid sequence set forth as one of SEQ ID NO: 1, the complement thereof, SEQ ID NO: 2 or SEQ ID NO: 3;
(ii) an amino acid sequence encoded by an open reading frame of a nucleic acid sequence at least 95% identical to the nucleic acid sequence set forth as one of SEQ ID NO: 1, the complement thereof, SEQ ID NO: 2 or SEQ ID NO: 3;
(iii) an amino acid sequence encoded by an open reading frame of the nucleic acid sequence set forth as one of SEQ ID NO: 1, the complement thereof, SEQ ID NO: 2 or SEQ ID NO: 3; or
(iv) an immunogenic fragment thereof,
or d) an antibody that specifically binds to the polypeptide of c); and
e) instructions for using the kit.

27-29. (canceled)

30. A method of producing an antibody, comprising
(a) immunizing a mammal with a polypeptide comprising:
(i) an amino acid sequence encoded by an open reading frame of a nucleic acid sequence at least 90% identical to the nucleic acid sequence set forth as one of SEQ ID NO: 1, the complement thereof, SEQ ID NO: 2 or SEQ ID NO: 3;
(ii) an amino acid sequence encoded by an open reading frame of a nucleic acid sequence at least 95% identical to the nucleic acid sequence set forth as one of SEQ ID NO: 1, the complement thereof, SEQ ID NO: 2 or SEQ ID NO: 3;
(iii) an amino acid sequence encoded by an open reading frame of the nucleic acid sequence set forth as one of SEQ ID NO: 1, the complement thereof, SEQ ID NO: 2 or SEQ ID NO: 3; or
(iv) an immunogenic fragment of the polypeptide;
or a nucleic acid encoding the polypeptide of (i)-(iv); and
(b) isolating an antibody that specifically binds
(i) the amino acid sequence encoded by an open reading frame of a nucleic acid sequence at least 90% identical to the nucleic acid sequence set forth as one of SEQ ID NO: 1, the complement thereof, SEQ ID NO: 2 or SEQ ID NO: 3;
(ii) the amino acid sequence encoded by an open reading frame of a nucleic acid sequence at least 95% identical to the nucleic acid sequence set forth as one SEQ ID NO: 1, the complement thereof, SEQ ID NO: 2 or SEQ ID NO: 3; or
(iii) the amino acid sequence encoded by an open reading frame of the nucleic acid sequence set forth as one of SEQ ID NO: 1, the complement thereof, SEQ ID NO: 2 or SEQ ID NO: 3;
thereby producing the antibody.

31. The method of claim 30, wherein the antibody specifically binds the amino acid sequence set forth as one of SEQ ID NOs: 4-7.

32. An isolated Phlebovirus comprising an S segment, an M segment and an L segment, wherein:
(i) the nucleotide sequence of the S segment is at least 80% identical to SEQ ID NO: 1;
(ii) the nucleotide sequence of the M segment is at least 80% identical to SEQ ID NO: 2;
(iii) the nucleotide sequence of the L segment is at least 80% identical to SEQ ID NO: 3; or
(iv) all (i), (ii) and (iii).

33-37. (canceled)

38. A Phlebovirus, wherein the Phlebovirus is attenuated, and wherein the Phlebovirus comprises an S segment, an M segment and an L segment, wherein:
the nucleotide sequence of the S segment is at least 80% identical to SEQ ID NO: 1;
(ii) the nucleotide sequence of the M segment is at least 80% identical to SEQ ID NO: 2;
(iii) the nucleotide sequence of the L segment is at least 80% identical to SEQ ID NO: 3; or
(iv) all (i), (ii) and (iii).

39. An immunogenic composition comprising an effective amount of the Phlebovirus of claim 32, an attenuated form of the Phlebovirus, an isolated polypeptide encoded by the Phlebovirus, a cDNA corresponding to an open reading frame of the Phlebovirus, or a vector comprising the cDNA, and a pharmaceutically acceptable carrier.

40. A method of eliciting an immune response against a Lone Star Virus in a subject, comprising administering to the subject a therapeutically effective amount of the immunogenic composition of claim 39, thereby eliciting the immune response to the Lone Star Virus.

41. The method of claim 40, wherein the subject is infected with the Lone Star Virus.

42. The method of claim 40, wherein the subject is healthy.

43. The method of claim 40, comprising administering to the subject the attenuated form of the Phlebovirus.
